(19) United States
(12) Patent Application Publication
(10) Pub. No.: US 2005/0288227 A1
(43) Pub. Date: Dec. 29, 2005

(54) Use of thioredoxin measurements for diagnostics and treatments

(76) Inventors: Paul A. Marks, Washington, CT (US); Johanna Ungerstedt, Stockholm (SE)

Correspondence Address: MINTZ LEVIN COHN FERRIS GLOVSKY & POPEO, 666 THIRD AVENUE, NEW YORK, NY, 10017, US

(21) Appl. No.: 11/144,301

(22) Filed: Jun. 3, 2005

Related U.S. Application Data
(63) Continuation-in-part of application No. 10/369,094, filed on Feb. 14, 2003, now abandoned.
(60) Provisional application No. 60/577,089, filed on Jun. 4, 2004.
(60) Provisional application No. 60/357,383, filed on Feb. 15, 2002.

(51) Int. Cl. A61K 38/00

(52) U.S. Cl. 514/12; 514/44

(57) ABSTRACT

The invention relates to methods for monitoring patient response to histone deacetylase inhibitors (e.g., suberoylanilide hydroxamic acid (SAHA)) or other therapeutic agents by measuring the level of thioredoxin in body fluids, tissues, and/or cells, such as peripheral blood mononuclear cells, plasma, or serum. The invention also relates to methods of monitoring and/or assisting with the diagnosis of a wide variety of thioredoxin-related diseases and conditions, such as inflammatory diseases, allergic diseases, autoimmune diseases, diseases associated with oxidative stress or diseases characterized by cellular hyperproliferation.

### RELATED APPLICATIONS

This application claims the benefit of U.S. Application No. 60/577,089 filed Jun. 4, 2004, and is a continuation-in-part of U.S. application Ser. No. 10/369,094 filed Feb. 14, 2003, which claims the benefit of U.S. Application No. 60/357,383 filed Feb. 15, 2002, all of which are hereby incorporated by reference herein in their entirety.

### GOVERNMENT SUPPORT

This invention was made at least in part with government support under NIH grants CA-0974823, UO1 CA-84292 and NCI Core Grant No. 08748. The government has certain rights in the invention.

### FIELD OF THE INVENTION

The invention relates to measurements of thioredoxin levels in biological samples. Specifically, the invention relates to methods for assessing levels of thioredoxin nucleic acids or thioredoxin polypeptides to monitor treatment with histone deacetylase inhibitors or other therapeutic agents and/or to monitor or assist in diagnosing a thioredoxin-related disorder.

### BACKGROUND OF THE INVENTION

The acetylation and deacetylation of histones play an important role in regulation of gene transcription (Grundstein M. Nature 389; 349-352, 1997). Histone acetylation is regulated by the opposing actions of histone acetyl transferases and histone deacetylases (HDACs; Marks P A et al., Nat Rev Cancer 1; 194-202, 2001). The HDAC inhibitor suberoylanilide hydroxamic acid (SAHA) has demonstrated its effectiveness against a broad variety of tumor cells in culture and in tumor bearing animal models (Richon, Proc Natl Acad Sci USA 95; 3003-3007, 1998). Inhibition of histone deacetylases by SAHA induces growth arrest, differentiation, and/or apoptosis of transformed cells in vitro (Richon V M et al., Proc Natl Acad Sci USA 93; 5705-5708, 1996), and inhibits tumor growth in vivo (Qiu L et al., Br J Cancer 80; 1252-1258, 1999; He L Z et al., J Clin Invest 108; 1321-1330, 2001). SAHA is currently in clinical trials and has shown significant antitumor activity in both solid and hematological tumors at doses well tolerated by patients (Kelly W K et al., Expert Opin Investig. Drugs 11; 1695-1713, 2002; Kelly W K et al., Clin Cancer Res 9; 3578-3588, 2003). However SAHA treatment may result in an attenuated or partial response, and, in certain cases, treatment can take several months to be observed. Therefore, there is a need in the art to quickly and easily determine patient response to SAHA and to determine the most effective therapeutic dose, since it has been shown that different patients may need different dosages for SAHA to achieve effectiveness.

Thioredoxin (TRX) is a 12 kDa ubiquitous multifunctional protein with the conserved active site sequence: -Cys-Gly-Pro-Cys- (SEQ ID NO:8) that forms a disulfide in the oxidized form or a dithiol in the reduced form. TRX plays an important biological role both in intra- and extracellular compartments. Researchers have reported that TRX is an intracellular redox protein with extracellular cytokine-like and chemokine-like activities (Nakamura, H. et al., Proc. Natl. Acad. Sci. USA, 98(5):2688-2693, 2001). This general protein dithiol-disulfide oxidoreductase can operate in a wide variety of intracellular processes either independently or together with NADPH and thioredoxin reductase (TR) as part of the TRX-TR system. In its reduced form, TRX is a hydrogen donor for ribonucleotide reductase essential for DNA synthesis and a general protein disulfide reductase involved in redox regulation. TRX plays an important role in the maintenance of an appropriate intracellular reduction/oxidation (redox) balance which is of crucial importance for normal cellular functioning that involves cell viability, signaling, activation, and proliferation. For example, TRX has been shown to be involved in the redox regulation of the transcription factors such as, NF-kappa-B and AP-1.

Abnormal levels of TRX have been found in numerous pathophysiological and disease states. For example, the expression of TRX can be enhanced by various types of stress, and as such, TRX is a stress-inducible protein. There has been accumulating evidence that TRX is induced and released from cells by a variety of oxidative stress conditions (Nakashima et al., Liver 2001, 21, 295-299 and references cited therein). TRX can behave as a scavenger of reactive oxygen intermediates (ROI), and as such, can offer protection against cytotoxicity, in which the generation of ROI can play a part in the cytotoxic mechanism. Recently it was reported that TRX induction in rats is accompanied with ROI overproduction and that TRX can play an important role not only in scavenging ROI but also in signal transduction during ischemia (Takagi et al., Neuroscience Letters (1998), 251, 25-28). It has recently been shown that serum levels of TRX in patients with heart failure is significantly higher than in control subject, indicating a possible association between TRX levels and the severity of heart failure (Kisimoto et al., Jpn. Cir. J. (2001), 65(6), 491-494). Increased plasma levels of thioredoxin were observed in patients with coronary spastic angina (Miyamoto et al., Antioxid Redox Signal 6:75-80, 2004).

Elevated levels of TRX have also been linked with chronic and/or malignant liver diseases. Researchers have reported that serum level of TRX is increased significantly in patients with hepatocellular carcinoma (Miyazaki et al., Oxid. Stress Dis. (1999), 3, 235-250). Furthermore, serum TRX levels have been found to be indicative of oxidative stress in patients with hepatitis C virus infection (Sumida Y, et al., J Hepatol 2000, 33:616-622). Various studies have reported that thioredoxin plasma/serum levels can be elevated under oxidative stress associated with viral infections, coronary spastic angina, and fatty liver disease (Nakamura, Nakamura H., 2004, Antioxidants and Redox Signaling 6, 15-17(3)). Elevated thioredoxin activity has also been observed in the cerebellum and cerebrum of mice exhibiting ataxia-telangiectasia, a disorder associated with oxidative stress (Kamsler et al., 2001, Cancer Res. 61:1849-1854).

Increased levels of TRX have also been found in cancer. TRX can stimulate proliferation of a wide variety of cancer cell lines and inhibit apoptosis in cells overexpressing the protein. In non-small cell lung cancer, overexpression of TRX is indicative of a more aggressive tumor phenotype and associated with bad prognostic features and possibly a poorer outcome (Kakolyris S et al., Clin Cancer Res 7(10); 3087-3091, 2001; Soini Y et al., Clin Cancer Res 7; 1750-1757, 2001). TRX is important for maintaining the growth of neoplastic cells, since it acts as a growth promoting factor. TRX exhibits anti-apoptotic effects by inhibiting ASK-1 and apoptosis induced by reactive oxygen species (Saitoh et al., EMBO J. 17; 2596-2606, 1998). TRX is also involved in inducing tumor cell resistance to several anti tumor drugs (Yokomizo et al., Cancer Res 55; 4293-4296, 1995). Thus, when TRX levels are elevated, there is increased tumor cell growth and resistance to the normal mechanisms of apoptosis.

In addition, TRX has recently been shown to be a potent chemotactic protein with potency comparable to other known chemokines, indicating a pathogenic role of TRX in infection and inflammation (Bertini, R. et al., J. of Exp. Med., 189(11):1783-1789, 1999). Since TRX production is induced by oxidants, a link between oxidative stress and inflammation is established. Indeed, TRX has been implicated in various inflammatory and autoimmune diseases. For example, it has been reported that the concentration of TRX in the synovial fluid and synovial tissue of patients suffering from rheumatoid arthritis (RA) is significantly increased and that based on the growth-promoting and cytokine-like properties the increased expression of TRX can contribute to the disease activity in RA (Maurice, M. et al., Arthritis & Rheumatism, 42(11):2430-2439, 1999). Furthermore, increased TRX levels have been reported in HIV disease (Nakamura et al., Int. Immunol. 8: 603-611, 1996).

A TRX-binding protein, designated as thioredoxin-binding protein-2 (TBP-2), was previously identified (Nishiyama, A. et al., J. Biol. Chem., 274(31):21645-50, 1999). TBP-2 is identical to vitamin D(3) up-regulated protein 1 (VDUP1). The association of TRX with TBP-2/VDUP1 was observed both in vitro and in vivo, showing that the TRX-TBP-2/VDUP1 interaction can affect the redox regulatory mechanism in cellular processes. In addition, it was shown that TBP-2/VDUP1 bound to reduced TRX but not to oxidized TRX. Importantly, it has been shown that both reducing activity and expression of TRX is inhibited by association with TBP-2. Thus, an induction in the expression of TBP-2 is associated with inhibition of both the biological function and expression of TRX. The ability of reduced TRX to inhibit apoptosis, and act as a growth factor, and the involvement of TRX in various disease states such as inflammatory and autoimmune diseases and conditions involving oxidative stress, indicate the need for methods of monitoring treatment and diagnosis of disorders characterized by altered levels of TRX.

### SUMMARY OF THE INVENTION

TRX protein levels in plasma are elevated in patients with various disorders, including inflammatory and autoimmune diseases and conditions involving oxidative stress or cellular proliferation. As demonstrated herein, SAHA (an HDAC inhibitor) causes a marked decrease in intracellular TRX protein levels in transformed cells. This decrease is associated with growth arrest and/or apoptosis of the transformed cells. In contrast, SAHA causes increases in the levels of intracellular TRX in non-transformed cells.

The present invention therefore utilizes the measurement of TRX levels in body fluids, tissues, or cells (e.g., blood plasma, serum, peripheral blood mononuclear cells, cancer cells, or tumor cells) to determine if a patient with a disorder/disease is responding to treatment with one or more HDAC inhibitors (e.g., SAHA) or other therapeutic agents. In responsive patients, TRX levels are predicted to decrease upon treatment. In non-responsive patients, TRX levels are predicted to remain elevated upon treatment. Dosage of an HDAC inhibitor or other therapeutic agent can be increased, maintained, or discontinued according to the results of these measurements.

The present invention also utilizes the measurement of TRX levels body fluids, tissues, or cells to monitor or assist in diagnosing a TRX-related disease or disorder. Where elevated TRX levels are associated wit a disease or disorder, the invention discloses methods for monitoring the condition and patient response to therapy.

In particular embodiments, the invention relates to methods for monitoring treatment with a HDAC inhibitor or other therapeutic agent comprising: (a) measuring levels of TRX in a biological sample from a patient undergoing treatment; (b) comparing the levels TRX in the biological sample to levels in a control sample (e.g., pre-treatment sample or other standard); and (c) determining if the levels of TRX in the biological sample are lower than the levels of TRX in the control sample. In certain aspects of the invention, the levels of TRX are determined using an antibody that binds to a TRX antigen. Preferably, the antibody is a monoclonal antibody and is, optionally, labeled. In other embodiments, the levels of TRX nucleic acids (e.g., mRNA transcripts) are determined using a nucleic acid probe or primers that binds to a TRX nucleotide sequence (e.g., mRNA). Preferably, the probe or primers comprise DNA and are, optionally, labeled.

In additional embodiments, the invention relates to methods for monitoring and/or assisting in the diagnosis of a TRX-related disease or disorder comprising: (a) measuring levels of TRX in a biological sample from a patient; (b) comparing the levels TRX in the biological sample to levels in a control sample (e.g., non-diseased sample or other standard); and (c) determining if the levels of TRX in the biological sample are altered (e.g., higher) the levels of TRX in the control sample. In certain aspects of the invention, the levels of TRX are determined using an antibody that binds to a TRX antigen. Preferably, the antibody is a monoclonal antibody and is, optionally, labeled. In other embodiments, the levels of TRX nucleic acid (e.g., mRNA transcripts) are determined using a nucleic acid probe or primers that binds to a TRX nucleotide sequence (e.g., mRNA). Preferably, the probe or primers comprise DNA and are, optionally, labeled.

In other embodiments, the invention relates to kits for determining TRX levels in a biological sample. In one aspect, the kit comprises one or more antibodies directed to a TRX antigen. Such kits can contain, for example, reaction vessels, reagents for detecting TRX in sample, and reagents for development of detected TRX, e.g. a secondary antibody coupled to a detectable marker. The label incorporated into the anti-TRX antibody may include, e.g., a chemiluminescent, enzymatic, fluorescent, colorimetric, or radioactive moiety. As an alternative approach, the kit can include one or more nucleic acid primers or probes for measuring levels of TRX gene expression. The nucleic acid primers or probes may be unlabeled or labeled with a detectable marker. If unlabeled, the nucleic acid primers or probes may be provided in the kit with labeling reagents. Such kits may be employed in diagnostic, monitoring, and/or clinical screening assays of the invention.

In specific aspects, the HDAC inhibitor or other therapeutic agent is directed to treatment of tumors, neoplasms, cancers, and other forms of cellular hyperproliferation, including, for example, gynecological neoplasms, central nervous system neoplasms, neoplasms of the head and neck, skin cancers, multiple endocrine neoplasia syndromes, tumors of the gastrointestinal tract, tumors of the lung, liver tumors, tumors of the bones and joints, AIDS-associated hematologic disorders and malignancies, thyroid cancers, breast cancers, genitourinary cancers, acute leukemias, chronic leukemias, lymphomas, and other conditions described in detail herein.

In particular aspects, the HDAC inhibitor includes, for example, (a) a hydroxamic acid derivative selected from SAHA, pyroxamide, CBHA, trichostatin A (TSA), trichostatin C, salicylihydroxamic acid (SBHA), azelaic bishydroxamic acid (ABHA), azelaic-1hydroxamate-9-anilid-e (AAHA), 6-(3-chlorophenylureido)carpoic hydroxamic acid (3Cl-UCHA), Oxamflatin, A-161906, Scriptaid, PXD-101, LAQ-824, CHAP, MW2796, and MW2996; (b) a cyclic tetrapeptide selected from, trapoxin A, FR901228 (FK 228, depsipeptide), FR225497, apicidin, CHAP, HC-toxin, WF27082, and chlamydocin; (c) a short chain fatty acid (SCFAs) selected from sodium butyrate, isovalerate, valerate, 4-phenylbutyrate (4-PBA), phenylbutyrate (PB), propionate, butyramide, isobutyramide, phenylacetate, 3-bromopropionate, tributyrin, valproic acid and valproate; (d) a benzamide derivative selected from CI-994, MS-27-275 (MS-275) and a 3′-amino derivative of MS-27-275; (e) an electrophilic ketone derivative selected from a trifluoromethyl ketone and an a-keto amide such as an N-methyl-a-ketoamide; and (f) depudecin, among others.

For other aspects of the invention, the therapeutic agent is an anti-cancer therapeutic that includes, e.g., radiation therapy, anthracyclines, flavopiridol, imatinib mesylate, retinoic acid, all-trans retinoic acid, demethylation agents, capecitabine, among others.

For certain aspects, the TRX-related disease or disorder includes, e.g., inflammatory diseases, autoimmune diseases, liver diseases, viral diseases, coronary disorders, disorders of cellular proliferation, and other conditions described in detail herein.

Other embodiments, objects, aspects, features, and advantages of the invention will be apparent from the accompanying description and claims.

### BRIEF DESCRIPTION OF THE DRAWINGS

FIG. 1. Schematic representation of a proposed model for the intracellular function of Thioredoxin (TRX). TRX is reduced by Thioredoxin reductase and NADPH. Reduced TRX (TRX(SH)₂) then reduces disulfides in target proteins such as transcription factors, receptors, and kinases, or provides reducing power for ribonucleotide reductase and TRX peroxidases. Reduced TRX binds to thioredoxin-binding protein-2 (TBP-2) and is then inactivated.

FIGS. 2A-2B. Suberoylanilide hydroxamic acid (SAHA) induces growth arrest in normal human lung fibroblasts (FIG. 2B), WI38, and transformed fibroblasts, VA13 (FIG. 2A). SAHA at doses of 2.5 pmol/l or more induces growth arrest of WI38 and VA13 cells.

FIGS. 3A-3B. SAHA induces apoptosis in transformed cells (FIG. 3A) but not in normal human lung fibroblasts (FIG. 3B). A striking difference in the sensitivity to SAHA-induced apoptosis was observed between normal and transformed cells. In transformed cells, 1.25-2.5 μmol/l SAHA was sufficient to induce significant apoptosis after 24-48 hours of SAHA treatment.

FIGS. 4A-4B. SAHA induces TRX mRNA and protein levels in cultured normal fibroblasts, but decreases TRX mRNA and protein levels in transformed cells. TRX and TPB-2 mRNA (FIG. 4A) and protein expression (FIG. 4B) in normal human fibroblasts, WI38, and in SV40 transformed human fibroblasts, VA13, cultured with 5 μmol/l SAHA.

FIGS. 5A-5F. TRX nucleotide and amino acid sequence information. FIG. 5A: GenBank Acc. Nos. J04026 (SEQ ID NO:1; SEQ ID NO: 9); FIG. 5B: GenBank Acc. No. BC003377 (SEQ ID NO:2; SEQ ID NO: 10); FIG. 5C: GenBank Acc. No. AF276919 (SEQ ID NO:3; SEQ ID NO:9); FIG. 5D: GenBank Acc. No. AY004872 (SEQ ID NO:4; SEQ ID NO: 10); FIG. 5E: GenBank Acc. No. AF313911 (SEQ ID NO:5; SEQ ID NO:10); FIG. 5F: GenBank Acc. No. BT007628 (SEQ ID NO:6; SEQ ID NO:10). Start and stop codons are shown in bold with underlining.

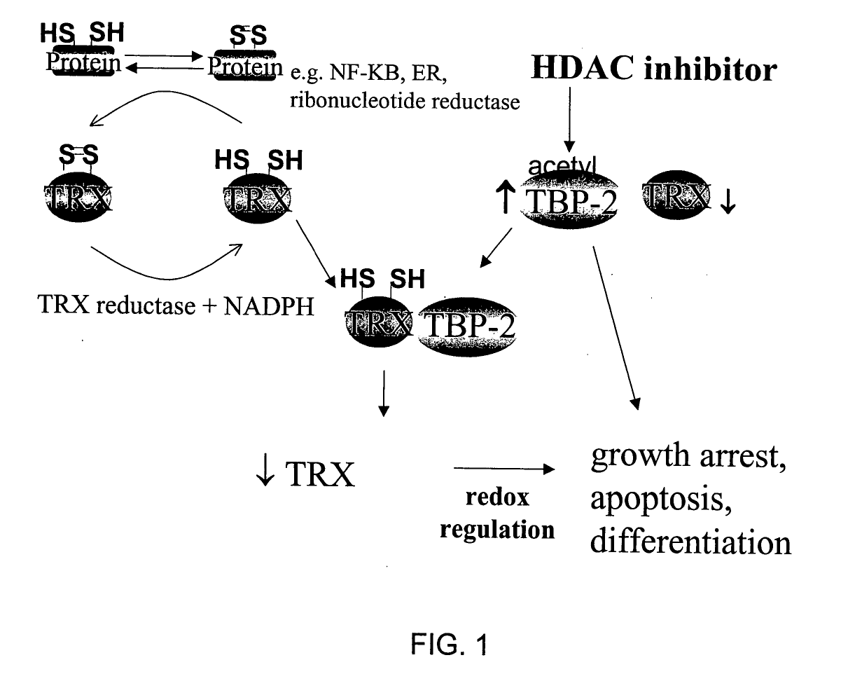

### DETAILED DESCRIPTION OF INVENTION

The invention provides techniques to monitor and/or assist with diagnosis of TRX-related diseases using measurements of TRX levels. The invention also provides minimally techniques to evaluate the biological activity of one or more HDAC inhibitors (e.g., SAHA) or other therapeutic agents used for treatment using measurements of TRX levels. This, in turn, can be used to infer the efficacy of such treatment. In one particular aspect of the invention, TRX levels can be measured in a biological sample (e.g., plasma, tumor cells, or cancer cells) from a patient with a disease/disorder before and during treatment with an therapeutic agent. A decrease of levels of TRX upon treatment would be indicative of the biological activity of the therapeutic agent. A partial decrease of levels of TRX upon treatment would be indicative of the need to increase dosage of the therapeutic agent, and thereby increase its biological activity. If TRX levels remain unchanged even at the higher dosage, it can be concluded that an alternate treatment agent should be pursued. It is an advantage of the invention that TRX plasma levels respond relatively rapidly in response to the HDAC inhibitor, SAHA, and precede any effects on tumor regression. Thus, the disclosed methods can be used to quickly determine the best candidates for SAHA treatment identify patients that would require alternative therapies. In one aspect of the invention, TRX levels in normal blood cells (e.g., peripheral blood mononuclear cells) may increase in patients who respond to SAHA.

Definitions

A “biological sample” for diagnostic, monitoring, or other clinical testing includes, but is not limited to, samples of blood (e.g., serum, plasma, whole blood, peripheral blood mononuclear cells (PBMCs), lymphocytes, or monocytes), nasal secretions, eye secretions, urine, fecal matter, saliva, sweat, breast milk, vaginal secretions, semen, cerebral spinal fluid, hair follicles, skin, teeth, bones, nails, cancer cells, tumor sample (e.g., biopsy), or other secretions, body fluids, tissues, or cells.

A “biological activity” of a therapeutic agent indicates, without limitation, an effect on one or more process (e.g., binding, signaling, oxidation, reduction, deacetylation, etc.), intracellular, intercellular, or extracellular, which can impact physiological or pathophysiological processes, especially cellular proliferation.

The term “treating” in its various grammatical forms in relation to the present invention includes preventing, curing, reversing, ameliorating, attenuating, alleviating, minimizing, suppressing or halting at least one deleterious symptom or effect of a disease or disorder state, or its progression, causative agent (e.g., bacteria or viruses), or other associated condition.

The term “antibody” as used herein refers to immunoglobulin molecules and immunologically active portions of immunoglobulin molecules, e.g., molecules that contain an antigen binding site that specifically binds (immunoreacts with) an antigen, such as a polypeptide or peptide. Such antibodies include, e.g., polyclonal, monoclonal, chimeric, single chain, Fab and F(ab′)₂ fragments, and components from an Fab expression library. In specific embodiments, antibodies are generated against human polypeptides or peptides, e.g., one or more TRX amino acid sequences.

The term “monoclonal antibody” or “monoclonal antibody composition”, as used herein, refers to a population of antibody molecules that contain only one species of an antigen binding site capable of immunoreacting with a particular epitope of a polypeptide or peptide. A monoclonal antibody composition thus typically displays a single binding affinity for a particular amino acid sequence with which it immunoreacts.

As used herein, specific forms of “cellular hyperproliferation” encompass tumors, neoplasms, and cancers, which include, but are not limited to, neuroblastoma, retinoblastoma, glioblastoma, basal cell carcinoma, melanoma, squamous cell carcinoma, merkel cell cancer, esophageal tumors, stomach cancer, small-bowel tumors, large-bowel tumors, pancreatic tumors, intracranial neoplasms, benign intracranial hypertension, spinal cord neoplasms, CNS paraneoplastic syndromes, cervical metastases, endometrial cancer, ovarian cancer, cervical cancer, vulvar cancer, vaginal cancer, fallopian tube cancer, gestational trophoblastic disease, thrombocytopenia, leukopenia, Kaposi's sarcoma, anal cancer, bronchiogenic carcinoma, benign breast disease (e.g., fibroadenoma, mastalgia, breast cysts, and gynecomastia), breast cancer (e.g., in situ carcinoma, ductal carcinoma, lobular carcinoma, cystosarcoma phyllodes, and invasive ductal and lobular tumors) Paget's disease, renal cell carcinoma, secondary renal cancer, cancer of the renal pelvis and ureter, bladder cancer, prostate cancer (e.g., adenocarcinoma of the prostate, undifferentiated prostate cancer, squamous cell carcinoma, and ductal transitional carcinoma of the prostate), urethral cancer, penile cancer, testicular cancer, acute lymphoblastic leukemia, acute myelogenous leukemia, chronic lymphatic leukemia, myelodysplastic syndrome, Hodgkin's disease, Non-Hodgkin's lymphomas, mycosis fungoides, and primary central nervous system lymphoma.

For the purposes of the invention, “TRX-related,” or “TRX-mediated,” or “TRX-involved” diseases/disorders are those that are correlated with abnormal levels (e.g., elevated transcript or polypeptide levels) of TRX, which include, but are not limited to disorders of cellular proliferation (see above); rheumatoid arthritis (RA); inflammatory conditions of the joint; psoriatic arthritis; inflammatory bowel diseases; spondyloarthropathies; scleroderma; psoriasis; inflammatory dermatoses; urticaria; vasculitis; eosinphilic myositis; eosinophilic fasciitis; cancers with leukocyte infiltration of the skin or organs; ischemic injury; cerebral ischemia; HIV; heart failure; coronary spastic angina; chronic, acute, or malignant liver disease; fatty liver disease; autoimmune thyroiditis; systemic lupus erythematosus; Sjorgren's syndrome; lung diseases; acute pancreatitis; ataxia-telangiectasia, amyotrophic lateral sclerosis (ALS); Alzheimer's disease; cachexia/anorexia; asthma; atherosclerosis; chronic fatigue syndrome; fever; diabetes; glomerulonephritis; graft versus host rejection; hemohorragic shock; hyperalgesia; multiple sclerosis; myopathies; osteoporosis; Parkinson's disease; pain; pre-term labor; psoriasis; reperfusion injury; cytokine-induced toxicity; side effects from radiation therapy; temporal mandibular joint disease; tumor metastasis; an inflammatory condition resulting from strain, sprain, cartilage damage, trauma, orthopedic surgery, infection or other disease processes; respiratory allergic diseases; systemic anaphylaxis; hypersensitivity responses; drug allergies and insect sting allergies.

The term “therapeutic agent” includes, but is not limited to, Aldesleukin, Alemtuzumab, alitretinoin, allopurinol, altretamine, amifostine, anastrozole, anthracyclines, arsenic trioxide, Asparaginase, BCG Live, bexarotene (e.g., capsules or gel), bleomycin, busulfan (e.g., intravenous or oral) calusterone capecitabine, carboplatin, carmustine (e.g. alone or with Polifeprosan 20 Implant), celecoxib, chlorambucil, cisplatin, cladribine, cyclophosphamide, cytarabine (e.g., liposomal), dacarbazine, dactinomycin/actinomycin D, Darbepoetin-alpha, daunorubicin (e.g., liposomal) daunorubicin/daunomycin, demethylation agents, Denileukin diftitox, dexrazoxane, docetaxel, doxorubicin (e.g., liposomal), dromostamolone propionate, Elliott's B Solution, epirubicin, Epoetin-alpha, estramustine, etoposide phosphate, etoposide VP-16, exemestane Filgrastim, flavopiridol, floxuridine (e.g., intraarterial), fludarabine, fluorouracil, 5-FU, fulvestrant, gemcitabine, gemcitabine, gemtuzumab, ozogamicin, goserelin acetate, hydroxyurea, Ibritumomab Tiuxetan, idarubicin, ifosfamide, imatinib mesylate, Interferon alpha-2a, Interferon alpha-2b, irinotecan, letrozole, leucovorin, levamisole, lomustine CCNU, meclorethamine/nitrogen mustard, megestrol acetate, melphalan/L-PAM, mercaptopurine/6-MP, mesna, methotrexate, methoxsalen, mitomycin C, mitotane, mitoxantrone, nandrolone, phenpropionate, Nofetumomab, Oprelvekin, oxaliplatin, paclitaxel, pamidronate, pegademase, Pegaspargase, Pegfilgrastim, pentostatin, pipobroman, plicamycin/mithramycin, porfimer sodium, procarbazine, quinacrine, radiation treatment (e.g., via implant, seed, or general irradiation), Rasburicase, retinoic acid, Rituximab, Sargramostim, streptozocin, talc, tamoxifen, temozolomide, teniposide/VM-26, testolactone, thioguanine/6-TG, thiotepa, topotecan, toremifene, Tositumomab, Trastuzumab, tretinoin/all-trans retinoic acid, Uracil Mustard, valrubicin, vinblastine, vincristine, vinorelbine, and zoledronate.

The phrase “SEQ ID NO:1-SEQ ID NO:6,” and the like, is used herein for convenience, and may refer to each SEQ ID NO individually or more than one SEQ ID NO in accordance with the methods of the invention.

The headings for the sections herein are provided for organizational purposes only. They are not to be considered limiting.

HDAC Inhibitors

The invention encompasses methods of utilizing TRX measurements to monitor treatment with one or more HDAC inhibitors (e.g., SAHA) or other therapeutic agents. HDAC inhibitors reported to date can be divided into several structural classes including: hydroxamates, cyclic peptides, aliphatic acids, benzamides, and electrophilic ketones (Table 1, below).

TABLE 1
HDAC inhibitors

| CLASS | COMPOUND | STRUCTURE |
|---|---|---|
| Hydroxamate | Trichostatin A (TSA) | 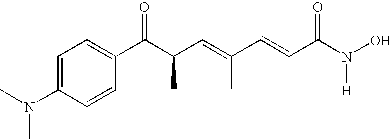 |
| | Suberoyl anilide hydroxamic acid (SAHA) | 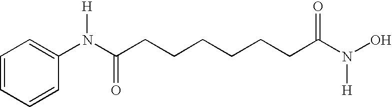 |
| Cyclic Peptide | Depsipeptide (FK-228) | 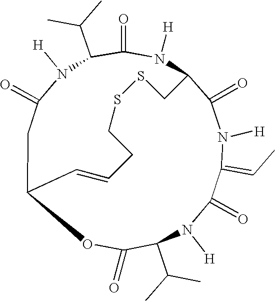 |
| Aliphatic Acid | Valproic Acid | 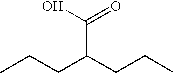 |
| | Phenyl Butyrate | 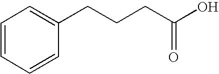 |
| Benzamide | MS-275 | 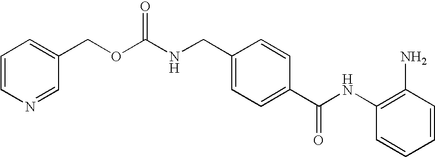 |
| | CI-994 | 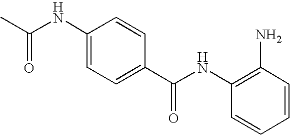 |
| Electrophilc Ketone | Trifluoromethyl Ketones | 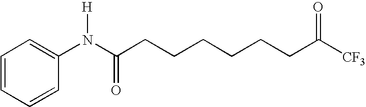 |
| | Alpha-ketoamides | 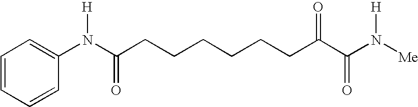 |

Trichostatin A (TSA) (Yoshida M, et al., J Biol Chem 1990; 265(28):17174-9) was the first natural product hydroxamate discovered to inhibit HDACs directly. Suberoylanilide hydroxamic acid (SAHA), which contains relatively less structural complexity, was found to be a nanomolar inhibitor of partially purified HDAC (Richon V M, et al., Proc Natl Acad Sci USA 1998; 95(6):3003-7). LAQ824 is another hydroxamic acid based analogue that is a nanomolar inhibitor of HDACs (Verdin E: Proceedings Novartis Foundation Symposium 2003; 259). Hydroxamic acid derivatives include, but are not limited to, SAHA (Richon et al., Proc. Natl. Acad. Sci. USA 95, 3003-3007 (1998)); m-carboxycinnanic acid bishydroxamide (CBHA) (Richon et al., supra); pyroxamide; CBHA; trichostatin analogues such as trichostatin A (TSA) and trichostatin C (Koghe et al. 1998. Biochem. Pharmacol. 56: 1359-1364); salicylihydroxamic acid (SBHA) (Andrews et al., International J Parasitology 30, 761-768 (2000)); azelaic bishydroxamic acid (ABHA) (Andrews et al., supra); azelaic-1-hydroxamate-9-anilide (AAHA) (Qiu et al., Mol. Biol. Cell 11, 2069-2083 (2000)); 6-(3-chlorophenylureido) carpoic hydroxamic acid (3Cl-UCHA), Oxamflatin ((2E)-5-[3-(phenylsulfonylamino)phenyl]pent-2-en-4-ynohydroxamic acid (Kim et al. Oncogene, 18: 2461 2470 (1999)); A-161906, Scriptaid (Su et al. 2000 Cancer Research, 60: 3137-3142); PXD-101 (Prolifix); LAQ-824; CHAP; MW2796 (Andrews et al., supra); and MW2996 (Andrews et al., supra).

Cyclic tetrapeptides, which constitute the most structurally complex class of HDAC inhibitors, encompass depsipeptide, apicidin, and the CHAPs molecules, which are active at nanomolar levels (Furumai R, et al., Cancer Res 2002; 62(17):4916-21; Singh S B, et al., J Org Chem 2002; 67(3):815-25; Furumai R, et al., Proc Natl Acad Sci USA 2001; 98(1):87-92). Cyclic tetrapeptides include, but are not limited to, trapoxin A (TPX)-cyclic tetrapeptide (cyclo-(L-phenylalanyl-L-phenylalanyl-D-pipecolinyl-L-2-amin-o-8-oxo-9,10-epoxydecanoyl)) (Kijima et al., J. Biol. Chem. 268, 22429-22435 (1993)); FR901228 (FK 228, Depsipeptide) (Nakajima et al., Ex. Cell Res. 241,126-133 (1998)); FR225497 cyclic tetrapeptide (H. Mori et al., PCT Application WO 00/08048 (Feb. 17, 2000)); apicidin cyclic tetrapeptide [cyclo (N O-methyl-L-tryptophanyl-L-isoleucinyl-D-pip-ecolinyl-L-2-amino-8oxodecanoyl)] (Darkin-Rattray et al., Proc. Natl. Acad. Sci. USA 93, 1314313147 (1996)); apicidin Ia, apicidin Ib, apicidin Ic, apicidin Ia, and apicidin IIb (P. Dulski et al., PCT Application WO 97/11366); CHAP, HC-Toxin cyclic tetrapeptide (Bosch et al., Plant Cell 7, 1941-1950 (1995)); WF27082 cyclic tetrapeptide (PCT Application WO 98/48825); and chlamydocin (Bosch et al., supra).

The aliphatic acids, the least potent class of HDAC inhibitors possess millimolar levels of activity, encompassing valproic acid (VA) and phenyl butyrate (PB) (Phiel C J, et al., J Biol Chem 2001; 276(39):36734-41; Boivin A J, et al., Anticancer Drugs 2002; 13(8):869-74). Short chain fatty acid (SCFA) derivatives include, but are not limited to, sodium butyrate (Cousens et al., J. Biol. Chem. 254, 1716-1723 (1979)); isovalerate (McBain et al., Biochem. Pharm. 53: 1357-1368 (1997)); Valerate (McBain et al., supra); 4-phenylbutyrate (4-PBA) (Lea and Tulsyan, Anticancer Research, 15,879-873 (1995)); phenylbutyrate (PB) (Wang et al., Cancer Research, 59, 2766-2799 (1999)); propionate (McBain et al., supra); butyramide (Lea and Tulsyan, supra); isobutyramide (Lea and Tulsyan, supra); phenylacetate (Lea and Tulsyan, supra); 3-bromopropionate (Lea and Tulsyan, supra); tributyrin (Guan et al., Cancer Research, 60,749-755 (2000)); valproic acid and valproate.

The benzamides, MS-275 and CI-994, are in general less potent than the corresponding hydroxamates and cyclic tetrapeptides (Prakash S, et al., Invest New Drugs 2001; 19(1):1-11; Saito A, et al., Proc Natl Acad Sci USA 1999; 96(8):4592-7). Benzamide derivatives include, but are not limited to, C₁₋₉₉₄; MS-27-275 [N-(2-aminophenyl)-4-[N-(pyridin-3-yl-methoxycarbonyl) aminomethyl] benzamide] (Saito et al., Proc. Natl. Acad. Sci. USA 96, 4592-4597 (1999)); and 3′-amino derivative of MS-27-275 (Saito et al., supra).

Electrophilic ketones belong to a new class of HDAC inhibitors and, like the benzamides, possess micromolar level inhibitory activities of HDAC. Electrophilic ketone derivatives include, but are not limited to, trifluoromethyl ketones (Frey et al., Bioorganic & Med. Chem. Lett. 2002, 12, 3443-3447; U.S. Pat. No. 6,511,990) and a-keto amides such as N-methyl-a-ketoamides. Other HDAC inhibitors include Depudecin (Kwon et al., 1998, Proc. Natl. Acad. Sci. USA 95: 3356-3361).

For use with the present invention, preferred HDAC inhibitors include Hydroxamic acid derivatives such as suberoylanilide hydroxamic acid (SAHA), m-carboxycinnamic acid bishydroxamate (CBHA) and pyroxamide or pharmaceutically acceptable salts or hydrates thereof. SAHA has been shown to bind directly in the catalytic pocket of the histone deacetylase enzyme. SAHA induces cell cycle arrest, differentiation, and/or apoptosis of transformed cells in culture and inhibits tumor growth in rodents. SAHA is effective at inducing these effects in both solid tumors and hematological cancers. It has been shown that SAHA is effective at inhibiting tumor growth in animals with no toxicity to the animal. The SAHA-induced inhibition of tumor growth is associated with an accumulation of acetylated histones in the tumor. SAHA is effective at inhibiting the development and continued growth of carcinogen-induced (N-methylnitrosourea) mammary tumors in rats. SAHA was administered to the rats in their diet over the 130 days of the study. Thus, SAHA is an orally active antitumor agent whose mechanism of action involves the inhibition of histone deacetylase activity and is well tolerated by patients.

Other examples of such compounds and other HDAC inhibitors can be found in U.S. Pat. No. 5,369,108, issued on Nov. 29, 1994, U.S. Pat. No. 5,700,811, issued on Dec. 23, 1997, U.S. Pat. No. 5,773,474, issued on Jun. 30, 1998, U.S. Pat. No. 5,932,616 issued on Aug. 3, 1999 and U.S. Pat. No. 6,511,990, issued Jan. 28, 2003 all to Breslow et al.; U.S. Pat. No. 5,055,608, issued on Oct. 8, 1991, U.S. Pat. No. 5,175,191, issued on Dec. 29, 1992 and U.S. Pat. No. 5,608,108, issued on Mar. 4, 1997 all to Marks et al.; as well as, Yoshida, M., et al., Bioassays 17, 423-430 (1995); Saito, A., et al., Proc. Natl. Acad. Sci. USA 96, 4592-4597, (1999); Furamai R. et al., Proc. Natl. Acad. Sci. USA 98 (1), 87-92 (2001); Komatsu, Y., et al., Cancer Res. 61(11), 4459-4466 (2001); Su, G. H., et al., Cancer Res. 60, 3137-3142 (2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 to Sloan-Kettering Institute for Cancer Research and The Trustees of Columbia University; published PCT Application WO02/246144 to Hoffmann-La Roche; published PCT Application WO02/22577 to Novartis; published PCT Application WO02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; and Curtin M. (Current patent status of histone deacetylase inhibitors Expert Opin. Ther. Patents (2002) 12(9): 1375-1384 and references cited therein).

Structural Formulas for HDAC Inhibitors

A wide variety of HDAC inhibitors are suitable for use in the compositions of the present invention. In a preferred embodiment, the HDAC inhibitor is suberoylanilide hydroxamic acid (SAHA), or a pharmaceutically acceptable salt or hydrate thereof, represented by the structure:

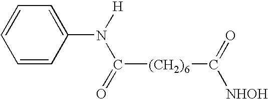

Other non-limiting examples of HDAC inhibitors that are suitable for use in the compositions of the present invention include the following.

Pyroxamide, or a pharmaceutically acceptable salt or hydrate thereof, represented by the structure:

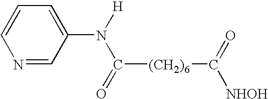

M-carboxycinnamic acid bishydroxamate (CBHA), or a pharmaceutically acceptable salt or hydrate thereof, represented by the structure:

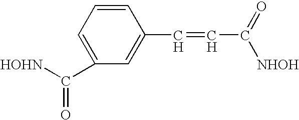

Additional HDAC inhibitors suitable for use with the invention can be represented by the structure: 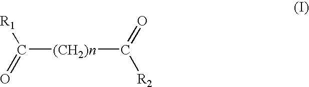
wherein R1 and R2 can be the same or different; when R₁ and R₂ are the same, each is a substituted or unsubstituted arylamino, cycloalkylamino, pyridineamino, piperamino, 9-purine-6-amino, or thiazoleamino group; when R₁ and R₂ are different R₁═R₃—N—R₄, where each of R₃ and R₄ are independently the same as or different from each other and are a hydrogen atom, a hydroxyl group, a substituted or unsubstituted, branched or unbranched alkyl, alkenyl, cycloalkyl, aryl, alkyloxy, aryloxy, arylalkyloxy, or pyridine group, or R₃ and R₄ are bonded together to form a piperidine group, R₂ is a hydroxylamino, hydroxyl, amino, alkylamino, dialkylamino, or alkyloxy group, and n is an integer from about 4 to about 8, or a pharmaceutically acceptable salt or hydrate thereof.

Additional examples of HDAC inhibitors suitable for use with the invention can be represented by the structure: 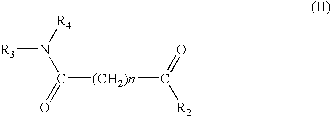
wherein R₃ and R₄ are independently a substituted or unsubstituted, branched or unbranched alkyl, alkenyl, cycloalkyl, aryl, alkyloxy, aryloxy, arylalkyloxy, or pyridine group, cycloalkyl, aryl, aryloxy, arylalkyloxy, or pyridine group, or R₃ and R₄ bond together to form a piperidine group; R₂ is a hydroxylamino group; and n is an integer from 5 to about 8, or a pharmaceutically acceptable salt or hydrate thereof.

In a particular embodiment of Formula I₁, R₂ is a hydroxylamino, hydroxyl, amino, methylamino, dimethylamino or methyloxy group and n is 6. In yet another embodiment of Formula II, R₄ is a hydrogen atom, R₃ is a substituted or unsubstituted phenyl, and n is 6. In further embodiments of Formula II, R₄ is hydrogen and R₃ is an α-, β-, or γ-pyridine.

In other specific embodiments of Formula II, R₄ is a hydrogen atom and R₃ is a cyclohexyl group; R₄ is a hydrogen atom and R₃ is a methoxy group; R₃ and R₄ each bond together to form a piperidine group; R₄ is a hydrogen atom and R₃ is a hydroxyl group; R₃ and R₄ are both a methyl group and R₃ is phenyl and R₄ is methyl.

Further HDAC inhibitors suitable for use with the invention can be represented by the structural formula: 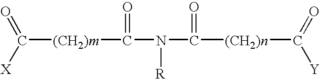
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamiho, aryloxyamino, alkyloxyalkylamino, or aryloxyalkylamino group; R is a hydrogen atom, a hydroxyl, group, a substituted or unsubstituted alkyl, arylalkyloxy, or aryloxy group; and each of m and n are independently the same as or different from each other and are each an integer from about 0 to about 8, or pharmaceutically acceptable salts or hydrates thereof.

In a particular embodiment, the HDAC inhibitor is a compound of Formula III wherein X, Y, and R are each hydroxyl and both m and n are 5.

In yet another embodiment, the HDAC inhibitor compounds suitable for use with the methods of the invention can be represented by the structural formula: 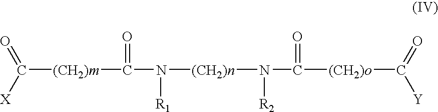
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m, n and o are independently the same as or different from each other and are each an integer from about 0 to about 8, or pharmaceutically acceptable salts or hydrates thereof.

Other HDAC inhibitors suitable for use with the invention include compounds having the structural formula: 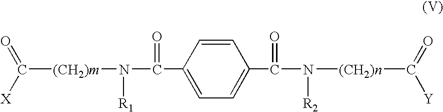
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m and n are independently the same as or different from each other and are each an integer from about 0 to about 8, or pharmaceutically acceptable salts or hydrates thereof.

In a further embodiment, HDAC inhibitors suitable for use with the invention can have structural formula: 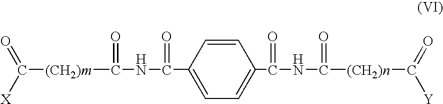
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; and each of m and n are independently the same as or different from each other and are each an integer from about 0 to about 8, or pharmaceutically acceptable salts or hydrates thereof.

In yet another embodiment, the HDAC inhibitors useful in the method of the invention can have structural formula: 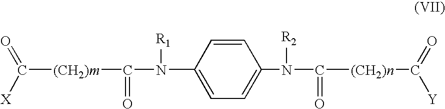
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; R₁ and R₂ are independently the same as or different from each other and are a hydrogen atom, a hydroxyl group, a substituted or unsubstituted alkyl, arylalkyloxy or aryloxy group; and each of m and n are independently the same as or different from each other and are each an integer from about 0 to about 8, or pharmaceutically acceptable salts or hydrates thereof.

In yet a further embodiment, HDAC inhibitors suitable for use in the invention can have the structural formula: 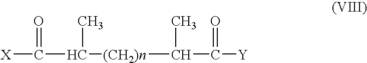
wherein each of X an Y are independently the same as or different from each other and are a hydroxyl, amino or hydroxylamino group, a substituted or unsubstituted alkyloxy, alkylamino, dialkylamino, arylamino, alkylarylamino, or aryloxyalkylamino group; and n is an integer from about 0 to about 8, or pharmaceutically acceptable salts or hydrates thereof.

Additional compounds suitable for use in the method of the invention include those represented by the structural formula: 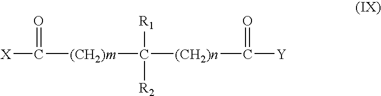
wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkyamino or aryloxyalkylamino group; each of R₁ and R₂ are independently the same as or different from each other and are a hydrogen atom, a hydroxyl group, a substituted or unsubstituted alkyl, aryl, alkyloxy, aryloxy, carbonylhydroxylamino or fluoro group; and each of m and n are independently the same as or different from each other and are each an integer from about 0 to about 8, or pharmaceutically acceptable salts and hydrates thereof.

In a further embodiment, HDAC inhibitors suitable for use in the invention include compounds having the structural formula: 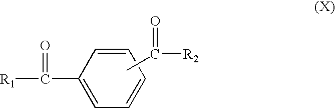
wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group. In a particular embodiment, the HDAC inhibitor is a compound of structural Formula X wherein R₁ and R₂ are both hydroxylamino groups, or pharmaceutically acceptable salts or hydrates thereof.

In a further embodiment, the HDAC inhibitors suitable for use in the invention have structural formula: 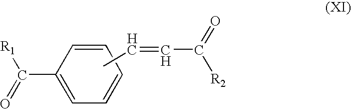
wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group or pharmaceutically acceptable salts or hydrates thereof. In a particular embodiment, the HDAC inhibitor is a compound of structural Formula XI wherein R₁ and R₂ are both hydroxylamino groups.

In a further embodiment, HDAC inhibitors suitable for use in the present invention include compounds represented by the structural formula: 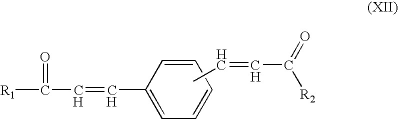
wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group or pharmaceutically acceptable salts or hydrates thereof. In a particular embodiment, the HDAC inhibitor is a compound of structural Formula XII wherein R₁ and R₂ are both hydroxylamino groups.

Additional compounds suitable for use in the method of the invention include those represented by the structural formula: 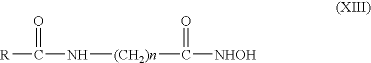
wherein R is a substituted or unsubstituted phenyl, piperidine, thiazole, 2-pyridine, 3-pyridine or 4-pyridine and n is an integer from about 4 to about 8, or a pharmaceutically acceptable salt or hydrate thereof.

In yet another embodiment, the HDAC inhibitors suitable for use in the method of the invention can be represented by the structural formula: 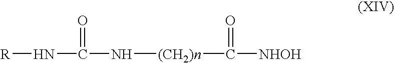
wherein R is a substituted or unsubstituted phenyl, pyridine, piperidine, or thiazole group and n is an integer from about 4 to about 8, or pharmaceutically acceptable salts or hydrates thereof. In a particular embodiment, R is phenyl and n is 5. In another embodiment, n is 5 and R is 3-chlorophenyl.

In structural formulas I-XIV, substituted phenyl, refers to a phenyl group which can be substituted with, for example, but not limited to a methyl, cyano, nitro, trifluoromethyl, amino, aminocarbonyl, methylcyano, halogen, e.g., chloro, fluoro, bromo, iodo, 2,3-difluoro, 2,4-difluoro, 2,5-difluoro, 3,4-difluoro, 3,5-difluoro, 2,6-difluoro, 1,2,3-trifluoro, 2,3,6-trifluoro, 2,3,4,5,6-pentafluoro, azido, hexyl, t-butyl, phenyl, carboxyl, hydroxyl, methyloxy, benzyloxy, phenyloxy, phenylaminooxy, phenylaminocarbonyl, methyloxycarbonyl, methylaminocarbonyl, dimethylamino, dimethylaminocarbonyl or hydroxyaminocarbonyl group.

Other HDAC inhibitors useful in the present invention can be represented by the structural formula: 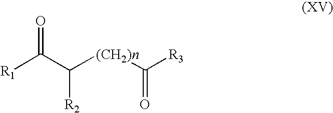
wherein each of R₁ and R₂ is directly attached or through a linker and is substituted or unsubstituted, aryl (e.g. naphthyl, phenyl), cycloalkyl, cycloalkylamino, pyridineamino, piperidino, 9-purine-6-amine, thiazoleamino group, hydroxyl, branched or unbranched alkyl, alkenyl, alkyloxy, aryloxy, arylalkyloxy, or pyridine group; n is an integer from about 3 to about 10 and R₃ is a hydroxamic acid, hydroxylamino, hydroxyl, amino, alkylamino or alkyloxy group, or pharmaceutically acceptable salts or hydrates thereof.

The linker can be an amide moiety, —O—, —S—, —NH— or —CH₂—. In certain embodiments, R₁ is —NH—R₄ wherein R₄ is substituted or unsubstituted, aryl (e.g., naphthyl, phenyl), cycloalkyl, cycloalkylamino, pyridineamino, piperidino, 9-purine-6-amine, thiazoleamino group, hydroxyl, branched or unbranched alkyl, alkenyl, alkyloxy, aryloxy, arylalkyloxy or pyridine group.

Further and more specific HDAC inhibitors within Formula XV, include those which can be represented by the formula: 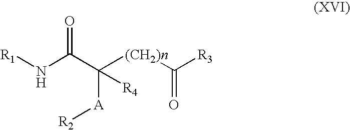
wherein each of R₁ and R₂ is, substituted or unsubstituted, aryl (e.g., phenyl, naphthyl), cycloalkyl, cycloalkylamino, pyridneamino, piperidino, 9-purine-6-amine, thiazoleamino group, hydroxyl, branched or unbranched alkyl, alkenyl, alkyloxy, aryloxy, arylalkyloxy or pyridine group; R₃ is hydroxamic acid, hydroxylamino, hydroxyl, amino, alkylamino or alkyloxy group; R₄ is hydrogen, halogen, phenyl or a cycloalkyl moiety; and A can be the same or different and represents an amide moiety, —O—, —S—, —NR₅— or —CH₂— where R₅ is a substitute or unsubstituted C₁-C₅ alkyl and n is an integer from 3 to 10, or pharmaceutically acceptable salts or hydrates thereof.

In addition, compounds having a more specific structure within Formula XVI can be represented by the structure: 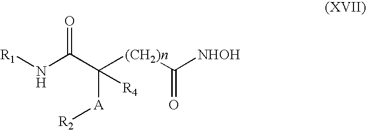
wherein A is an amide moiety, R1 and R2 are each selected from substituted or unsubstituted aryl (e.g., phenyl, naphthyl), pyridineamino, 9-purine-6-amine, thiazoleamino, aryloxy, arylalkyloxy or pyridine and n is an integer from 3 to 10 or pharmaceutically acceptable salts or hydrates thereof.

For example, the compound can have the formula:

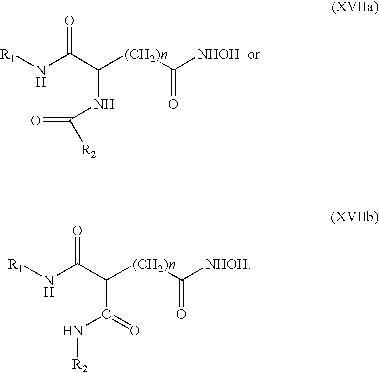

In another embodiment, the HDAC inhibitor can have the formula: 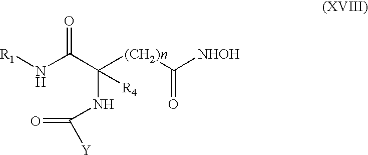
wherein R₇ is selected from substituted or unsubstituted aryl (e.g., phenyl or naphthyl), pyridineamino, 9-purine-6-amine, thiazoleamino, aryloxy, arylalkyloxy or pyridine and n is an integer from 3 to 10 and Y is selected from:

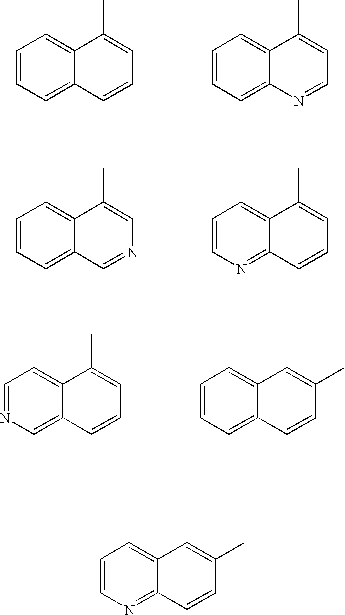

Also included are pharmaceutically acceptable salts or hydrates thereof.

In a further embodiment, the HDAC inhibitor compound can have the formula: 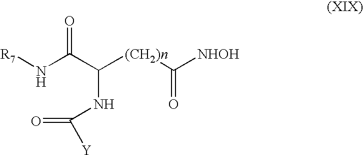
wherein n is an integer from 3 to 10, Y is selected from: 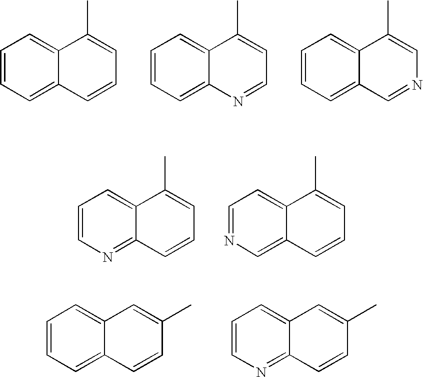
and R₇ is selected from: 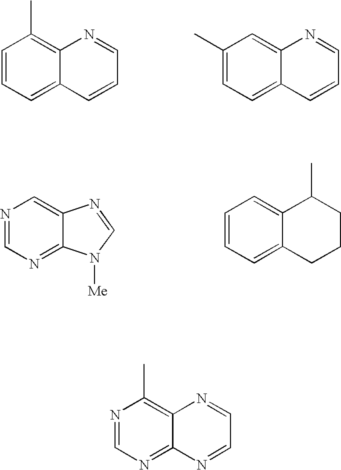
Also included are pharmaceutically acceptable salts or hydrates thereof.

Further compounds for use in the invention can be represented by the structural formula: 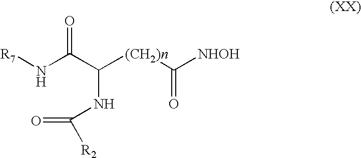
wherein R₂ is selected from substituted or unsubstituted aryl, substituted or unsubstituted naphtha, pyridineamino, 9-purine-6-amine, thiazoleamino, substituted or unsubstituted aryloxy, substituted or unsubstituted arylalkyloxy or pyridine and n is an integer from 3 to 10 and R₇ is selected from: 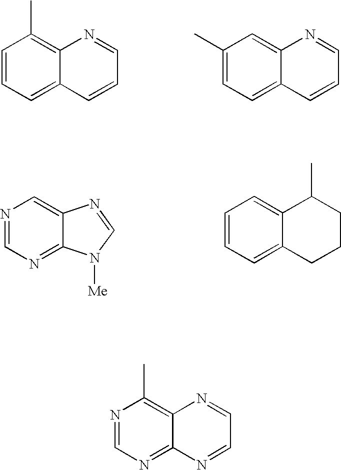
Also included are pharmaceutically acceptable salts or hydrates thereof.

Further HDAC inhibitors useful in the invention can be represented by the structural formula: 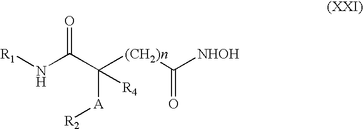
wherein A is an amide moiety, R₁ and R₂ are each selected from substituted or unsubstituted aryl, naphtha, pyridineamino, 9-purine-6-amine, thiazoleamino, aryloxy, arylalkyloxy or pyridine, R₄ is hydrogen, a halogen, a phenyl or a cycloalkyl moiety and n is an integer from 3 to 10, or pharmaceutically acceptable salts or hydrates thereof.

For example, a compound of Formula XXI can be represented by the structure: 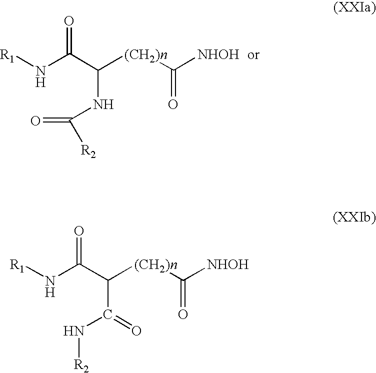
wherein R₁, R₂, R₄, and n have the meanings of Formula XXI, or pharmaceutically acceptable salts or hydrates thereof.

Further, HDAC inhibitors can include those having the structural formula: 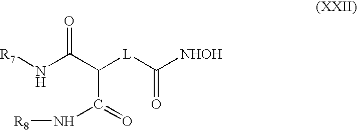
wherein L is a linker selected from the group consisting of —(CH₂)n—, —(CH═CH)m, phenyl, -cycloalkyl-, or any combination thereof; and wherein each of R₇ and R₈ are independently substituted or unsubstituted, aryl, naphtha, pyridineamino, 9-purine-6-amine, thiazoleamino group, aryloxy, arylalkyloxy, or pyridine group, n is an integer from 3 to 10 and m is an integer from 0 to 10, or pharmaceutically acceptable salts or hydrates thereof.

For example, a compound of Formula XXII can be:

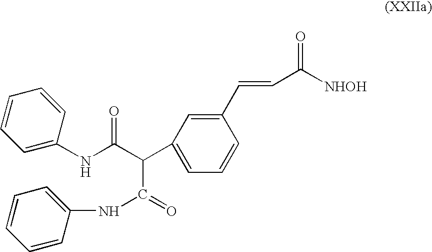

Other HDAC inhibitors suitable for use in the invention include those shown in the following more specific formulas: 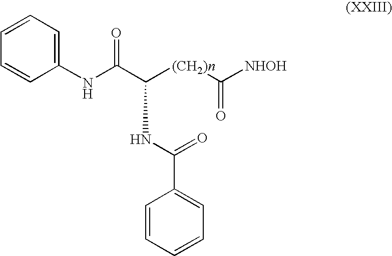
wherein n is an integer from 3 to 10 or an enantiomer, or: 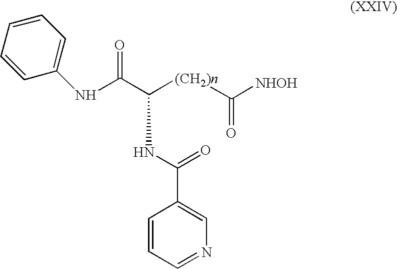
wherein n is an integer from 3 to 10 or an enantiomer, or: 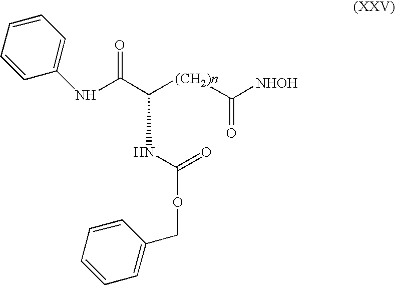
wherein n is an integer from 3 to 10 or an enantiomer, or: 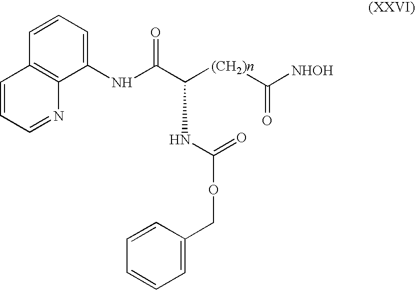
wherein n is an integer from 3 to 10 or an enantiomer, or: 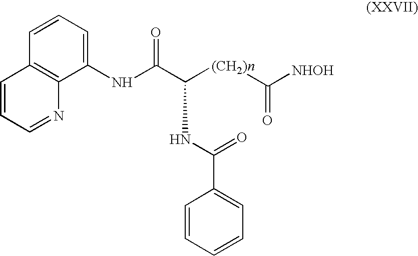
wherein n is an integer from 3 to 10, or an enantiomer, or pharmaceutically acceptable salts or hydrates of all of the above.

Further specific HDAC inhibitors suitable for use in the invention include: 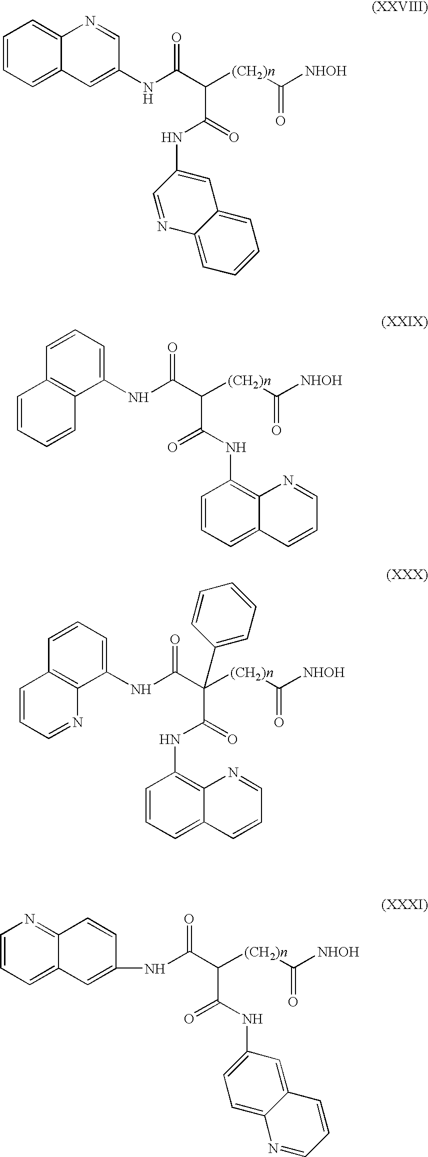
wherein n in each is an integer from 3 to 10, or pharmaceutically acceptable salts or hydrates of all of the above, and the compound: 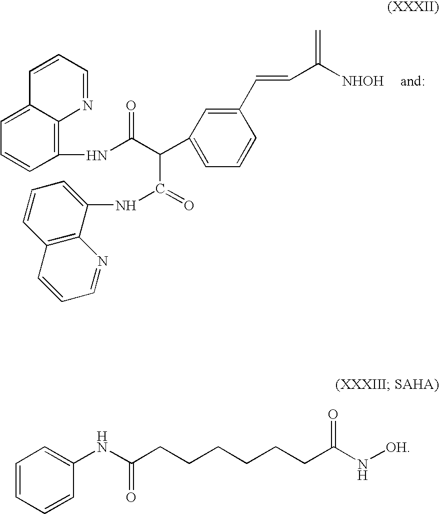
HDAC Inhibitor-Based Treatments

The invention specifically encompasses methods of utilizing TRX measurements to monitor treatments with one or more HDAC inhibitors (e.g., SAHA) or other therapeutic agents for cellular hyperproliferation (e.g., tumors, neoplasms, or cancers), autoimmune diseases, inflammatory disorders, or oxidative stress disorders. HDAC inhibitors fall into a class of agents that target an activity (reversible protein acetylation) that occurs in all cells rather than targeting an abnormal process in the cancer cell. The favorable therapeutic index observed in tumor bearing animal studies and in clinical trials (see below) appears to result from the differential response of the cancer cells and normal cells to inhibition of HDAC activity. This type of selectively takes advantage of the contextual differences between normal and cancer cells, and has been previously described for the proteosome inhibitor, MLN341 and the heat-shock inhibitor, geldanamycin (Reddy A, et al., Curr Opin Pharmacol 2002; 2(4):366-73).

HDAC inhibitors have been found to be synergistic or additive with a number of anti-cancer agents, including radiation, anthracyclines, flavopiridol, Gleevec (imatinib mesylate) and all-trans retinoic acid (Marks P, et al., Nat Rev Cancer 2001; 1(3):194-202, Marks P, et al., Current Opinion in Pharmacology 2003, 3:344-351; Johnstone R W, et al., Cancer Cell 2003; 4(1):13-8) in tumor cells in culture. HDAC inhibitors may act synergistically or additively with agents that act on DNA (e.g. radiation and anthracyclines) by altering the conformation of chromatin resulting in a more open structure allowing access to DNA and increased ability to cause damage to DNA. HDAC inhibitors may alter gene expression that then cause cells to become more sensitive to the agents, such as Gleevec and flavopiridol. The combination of HDAC inhibitor and an inhibitor of protein regulation cell cycle progression, such as, flavopiridol, may synergistic owning to targeting multiple sites of aberrant function in cancer cells.

A number of HDAC inhibitors are in clinical trials. Only phenylacetate (PA) is approved for human use in the treatment of urea cycle disorders in children, portal encephalopathy, and chemotherapy-induced hyperammonemia. In patients with malignant diseases, PA demonstrated modest palliative benefits (Chang S M, et al., J Clin Oncol 1999; 17(3):984-90). Phenylbutyrate (PB) is a precursor of phenylacetate after β-oxidization in the liver and kidney. PB has been shown to inhibit histone acetylation, modify lipid metabolism and activate peroxisome proliferation activator receptor. Studies used a prolonged intravenous infusion (Gilbert J, et al., Clin Cancer Res 2001; 7(8):2292-300). Modest clinical activity was documented in patients with leukemia, myelodysplastic syndrome, and several solid tumors.

An oral formulation of PB has been tested that showed that it had good bioavailability and was able to reach biologic active plasma concentrations (0.5 mM). Clinical studies with PB have shown prolonged stabilization of the disease in several patients suggesting cytostatic effects. In one report, PB was combined with all-trans retinoic acid (ATRA), which restored the sensitivity to ATRA in patients with acute promyelocytic leukemia (APL) that progressed after ATRA treatment, alone (Kelly W K, et al., Expert Opin Investig Drugs 2002; 11(12):1695-1713). It is postulated that PB inhibits the co-repressor complex that contains HDAC for the oncoprotein that is encoded by one of the translocation-generated fusion genes in APL, PML-RA Rα. Other trials using retinoic acid and demethylation agents (e.g., 5-azacytidine (5-AzaC) and 5-deoxy-azacytidine (DAC)) are ongoing to exploit the cell modulating effects of phenylbutyrate.

Valproic acid (VA) is a short chain fatty acid that is a well tolerated antiepileptic agent and that has recently been shown to be an inhibitor of HDACs (Gottlicher M, et al., EMBO J. 2001; 20(24):6969-78). In a clinical trial in a patient with acute myelogenous leukemia, VA induced a transient partial remission. Ongoing clinical trials are further evaluating VA into patients with hematologic and solid tumor.

Other investigational histone deacetylase inhibitors are undergoing clinical evaluation. These include the cyclic peptide, depsipeptide; the hydroxamic acid-based HDAC inhibitors, SAHA; pyroxamide and LAQ824; the benzamide, MS-275; and N-acetyl amide (CI-994) that inhibits HDAC by an undetermined mechanism.

Depsipeptide is isolated from Chromobacterium violaceum and inhibits HDACs at nanomolar concentrations. Few cardiac events were seen in the initial phase I study. These trials established the biologic activity of depsipeptide by showing an increased accumulation of acetylated histones in post-treatment samples of PMN cells. An anti-tumor effect was seen in patients with renal cell carcinoma and in a patient with T-cell lymphomas. Phase II clinical trial is ongoing with depsipeptide (Piekarz R L, Robey R, Bakke S, Sandor V, Wilson W, Bates S ASCO 2001:232b).

SAHA and Pyroxamide are two potent hydroxamic acid based inhibitors of HDACs. Phase I studies with intravenously administered SAHA showed that this novel agent will cause the accumulation of acetylated histones in normal and malignant cells and anti-tumor effects were seen in patient with solid and hematological tumors (Kelly W K, et al., Clinical Cancer Research 2003, 9, 3578-3588). An oral formulation of SAHA has been under clinical development that has shown good oral bioavailibity and favorable pharmakinetic profile (Kelly W K, et al., 14th EORTC-NCI-AACR, Frankfurt 2002). Following a single oral dose of SAHA, accumulation of acetylated histones in PMN cells can persist for up to 10 hrs. Patients with renal cell carcinoma, squamous cell carcinoma of the head and neck, papillary thyroid cancer, mesothelioma, B- and T-cell lymphomas and Hodgkin's disease have shown clinical improvement on oral SAHA. There have been durable responses with the longest duration of therapy extending to over 18 months. There have been no drug related deaths. The dose limiting toxicities have been non-hematologic. All toxicities have been reversible on cessation of drug administration. SAHA is under evaluation in Phase II clinical trials.

MS-275 is a potent inhibitor of HDAC that has entered into phase I clinical trials. Initial daily dosing schedules was poorly tolerated, probably due to the longer than expected half-life of the drug. Alternative dosing schedules have been better tolerated and preliminary results suggest an in vivo effect with an increase in acetylated histones in PMN cells and an induction of apoptosis in leukemic cells.

CI-994 (N-acetylamide) is an orally bioavailable compound that has been shown to cause phosphorylation and degradation of nuclear proteins with subsequent accumulation of acetylated histones in malignant cell lines. For CI-994, cytostatic effects are seen in multiple solid tumor cell lines. The oral preparation is well tolerated. Phase II studies in patients with non-small cell lung cancer showed minimal clinical activity with two out of 32 (7%) patients having a partial response to therapy and 28% of the patients having stable disease for over 8 weeks (Wozniak A, et al., ASCO 1999:487a). In Phase II studies in patients with metastatic renal cell carcinoma, stable disease for greater than 8 weeks was documented in 58% of the patients. CI-994 was combined with capecitabine, which lead to significant thrombocytopenia and hand-foot syndrome at the highest doses with partial response reported in one patient with colorectal cancer.

Nucleic Acids

In accordance with the invention, the measurement of TRX levels can be determined using a nucleic acid probe that binds to a TRX nucleotide sequence. Nucleic acid molecules of the invention can be RNA (e.g., mRNA transcripts), or DNA, such as cDNA and genomic DNA. DNA molecules can be double-stranded or single-stranded; single stranded RNA or DNA can be either the coding (sense) strand or the non-coding (antisense) strand. Preferably, the nucleic acid molecule comprises at least 15 contiguous nucleotides, at least 30 contiguous nucleotides, at least 60 contiguous nucleotides, at least 100 contiguous nucleotides, at least 150 contiguous nucleotides, or at least contiguous 300 nucleotides of a TRX nucleotide sequence (e.g., SEQ ID NO:1-SEQ ID NO:6 or a complementary sequence thereof). The nucleic acid molecule can encodes at least a fragment of the amino acid sequence of the TRX polypeptide (e.g., SEQ ID NO:9-SEQ ID NO:10). Alternatively, the nucleic acid molecule can include at least a fragment with non-coding sequences such as introns and non-coding 3′ and 5′ sequences, including regulatory sequences, for example.

As used herein, an “isolated” or “substantially pure” nucleic acid molecule is intended to mean a nucleotide sequence, which has been completely or partially purified from other transcribed, replicated, or chemically synthesized sequences. Thus, an isolated nucleotide sequence can include a nucleotide sequence, which is synthesized chemically or by recombinant means. Thus, recombinant DNA contained in a vector is included in the definition of “isolated” as used herein. Also, isolated nucleotide sequences include recombinant DNA molecules in heterologous host cells, as well as partially or substantially purified DNA molecules in solution. In vivo and in vitro RNA transcripts of the DNA molecules of the present invention are also encompassed by “isolated” nucleotide sequences. Such isolated nucleotide sequences can be particularly useful for detecting expression of TRX in biological samples (e.g., blood plasma, serum, or PBMC), such as by Northern blot or dot blot analysis or RT-PCR.

In certain embodiments, TRX nucleotide sequences can include those previosuly published (e.g., Wollman et al., J. Biol. Chem. 263 (30), 15506-15512 (1988); Strausberg et al., Proc. Natl. Acad. Sci. USA 99 (26), 16899-16903 (2002); Yegorova et al., Invest. Ophthalmol. Vis. Sci. 44 (8), 3263-3271 (2003)). The present invention also pertains to nucleic acid molecules that comprise a sequence that is different from the naturally-occurring nucleotide sequence but which, due to the degeneracy of the genetic code, encode at least a fragment of a TRX polypeptide (e.g., SEQ ID NO:9-SEQ ID NO:10). The invention also encompasses variations of the nucleotide sequences of the invention, such as those encoding portions, analogues or derivatives of at least a fragment of a TRX polypeptide. Such variations can be naturally-occurring, such as in the case of allelic variation, or non-naturally-occurring, such as those induced by various mutagens and mutagenic processes. Intended variations include, but are not limited to, addition, deletion, and substitution of one or more nucleotides, which can result in conservative or non-conservative amino acid changes, including additions and deletions.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Non-limiting examples of useful labels include radiolabels (e.g., ³²P, ¹⁴C, ³H), enzymes (e.g., horseradish peroxidase, alkaline phosphatase, β-galactosidase, or acetylcholinesterase), fluorescent compounds (e.g., umbelliferone, fluorescein, fluorescein isothiocyanate, rhodamine, dichlorotriazinylamine fluorescein, dansyl chloride or phycoerythrin), luminescent or bioluminescent compounds, streptavidin, avidin, biotin, magnetic moieties, metal-binding moieties, digoxigenin, antigen or antibody moieties, and the like. Also encompassed are synthetic molecules that mimic nucleic acid molecules in the ability to bind to designated sequences via hydrogen bonding and other chemical interactions. Such molecules include, for example, those in which peptide linkages substitute for phosphate linkages in the backbone of the molecule.

The invention also relates to fragments of the isolated nucleic acid molecules described herein. The term “fragment” is intended to encompass a portion of a nucleic acid: sequence described herein, such as a portion that encodes a fragment of a TRX polypeptide. For example, a fragment can be a portion of a nucleic acid (e.g., SEQ ID NO:1-SEQ ID NO:6 or a complementary sequence thereof) that is from at least 15 contiguous nucleotides to at least 300 contiguous nucleotides or longer in length. One or more introns can also be present. Such fragments are useful as probes, e.g., for diagnostic methods and also as primers (e.g., PCR primers) or probes. Particularly preferred primers and probes selectively hybridize to the nucleic acid molecule encoding a TRX polypeptide.

The invention also pertains to nucleic acid molecules that hybridize under medium, and, more preferably, high, stringency hybridization conditions (e.g., for selective hybridization) to a portion of a nucleic acid molecule described herein. Appropriate stringency conditions are known to those skilled in the art or can be found in standard texts such as Current Protocols in Molecular Biology, John Wiley & Sons, New York (1998), 6.3.1-6.3.6. Such hybridizable nucleic acid molecules are useful as probes and primers for diagnostic or monitoring applications. For example, high stringency hybridization conditions for Northern blotting include conditions with a temperature that is from about 68° C. below the calculated Tm (Tm is based upon the nucleotide sequence of the probe and can be calculated for each probe); alternatively, high stringency conditions include low salt conditions.

Accordingly, the invention pertains to nucleic acid molecules that have a substantial identity with the nucleotide sequences described herein. Particularly preferred are nucleic acid molecules that have at least 60%, more preferably at least 85%, even more preferably at least 95%, and still more preferably at least 99% identity with nucleotide sequences described herein. For example, preferred nucleic acid molecules encoding a TRX polypeptide having the same or similar immunogenic or antigenic properties as the naturally occurring TRX polypeptide are within the scope of the invention. Nucleic acid molecules that have lower overall homologies are also included herein, provided that they have substantial identity over fragments of the polypeptide. Thus, nucleic acid molecules which similarly have lower overall homology to a TRX polypeptide, but which have substantial homology to one or more regions of the TRX polypeptide, are encompassed by the invention.

The invention also provides expression vectors containing at least a fragment of a TRX nucleic acid sequence (e.g., SEQ ID NO: 1-SEQ ID NO:6), operably linked to at least one regulatory sequence. Many such vectors are commercially available, and other suitable vectors can be readily prepared by the skilled artisan. “Operably linked” is intended to mean that the nucleotide sequence is linked to a regulatory sequence in a manner that allows expression of the nucleotide sequence. Accordingly, the term “regulatory sequence” includes promoters, enhancers, and other expression control elements that are described in Goeddel, Gene Expression Technology: Methods in Enzymology 185, Academic Press, San Diego, Calif. (1990). For example, the native regulatory sequences or regulatory sequences native to the transformed host cell can be employed.

It should be understood that the design of the expression vector may depend on such factors as the choice of the host cell to be transformed and/or the type of protein desired to be expressed. For instance, the polypeptides encoded by the nucleic acid molecules of the present invention can be produced by ligating the cloned gene, or a portion thereof, into a vector suitable for expression in either prokaryotic cells, eukaryotic cells or both (see, for example, Broach, et al., Experimental Manipulation of Gene Expression, ed. M. Inouye (Academic Press, 1983) p. 83; Molecular Cloning: A Laboratory Manual, 2nd Ed., ed. Sambrook et al. (Cold Spring Harbor Laboratory Press, 1989) Chapters 16 and 17). Typically, expression constructs will contain one or more selectable markers, including, but not limited to, the gene that encodes dihydrofolate reductase and the genes that confer resistance to neomycin, tetracycline, ampicillin, chloramphenicol, kanamycin and streptomycin resistance. Vectors can also include, for example, an autonomously replicating sequence (ARS), expression control sequences, ribosome-binding sites, RNA splice sites, polyadenylation sites, transcriptional terminator sequences, secretion signals, and mRNA stabilizing sequences.

Prokaryotic and eukaryotic host cells transformed by the described vectors are also provided by this invention. For instance, cells which can be transformed with the vectors of the present invention include, but are not limited to, bacterial cells such as E. coli (e.g., E. coli K12 strains), Streptomyces, Pseudomonas, Serratia marcescens and Salmonella typhimurium, insect cells (e.g., baculovirus), including Drosophila, fungal cells, such as yeast cells (e.g., Saccharomyces cerevisiae), plant cells and mammalian cells, such as thymocytes, Chinese hamster ovary cells (CHO), and COS cells. The host cells can be transformed by the described vectors by various methods (e.g., electroporation, transfection using calcium chloride, rubidium chloride, calcium phosphate, DEAE-dextran, or other substances; microprojectile bombardment; lipofection, infection where the vector is an infectious agent such as a retroviral genome, and other methods), depending on the type of cellular host.

The nucleic acid molecules of the present invention can be produced, for example, by replication in a suitable host cell, as described above. Alternatively, the nucleic acid molecules can also be produced by chemical synthesis or PCR-based techniques.

Polypeptides

In accordance with the invention, the measurement of TRX levels can be determined using an antibody that binds to a TRX polypeptide or peptide sequence. The term “polypeptide” refers to a polymer of amino acids, and not to a specific length; thus, peptides, oligopeptides, and proteins are included within the definition of a polypeptide. A polypeptide that “shares significant identity” with is a polypeptide that has approximately 75% amino acid identity with TRX (e.g., SEQ ID NO:9-SEQ ID NO:10). Polypeptides exhibiting lower levels of identity are also useful and can be considered to be TRX polypeptides, particular if they exhibit high, e.g., at least 80%, more preferably at least 90%, and even more preferably at least 95%, amino acid identity with TRX over one or more particular domains of the polypeptide (e.g., SEQ ID NO:8). For example, polypeptides sharing high degrees of identity over domains necessary for particular activities, including antibody binding activity, are included herein.

A TRX polypeptide of the present invention can be isolated or purified (e.g., to homogeneity) following recombinant methods or other synthesis methods by a variety of processes. A polypeptide that is “isolated” is substantially free of associated components, such as by separation from the components which accompany it in its natural state or in synthesis systems. Thus, a polypeptide that is chemically synthesized, or synthesized in a cellular or cell-free system, will be substantially free of naturally associated components, and thus, is considered to be “isolated”. Methods of isolation include, but are not limited to, anion or cation exchange chromatography, ethanol precipitation, polyacrylamide gel electrophoresis, affinity chromatography and high performance liquid chromatography (HPLC). The particular method used will depend upon the properties of the polypeptide or peptide and the selection of the synthesis method. Appropriate methods will be readily apparent to those skilled in the art.

According to the invention, the amino acid sequence of the TRX polypeptide can be that of the naturally-occurring polypeptide or can comprise alterations therein. Such alterations include conservative or non-conservative amino acid substitutions, additions, and deletions of one or more amino acids; however, such alterations should preserve at least one activity of the TRX polypeptide. For example, the mutation(s) can preferably preserve the three dimensional configuration of an antibody binding site of the native polypeptide. Alternatively, the fragment retains other immunological activities, such as immunogenic function, as well as sharing of immunological epitopes for binding.

The presence or absence of TRX polypeptide activity can be determined by various standard functional assays including, but not limited to, assays for binding of anti-TRX antibodies to the polypeptide. Moreover, amino acids that are essential for the function of a TRX polypeptide can be identified by methods known in the art. Particularly useful methods include identification of conserved amino acids in the superfamily of immunoglobulin genes, site-directed mutagenesis and alanine-scanning mutagenesis (for example, see, Cunningham and Wells, Science 244:1081-1085 (1989)), crystallization and nuclear magnetic resonance. The altered polypeptides produced by these methods can be tested for particular biologic activities, including immunogenicity and antigenicity.

Specifically, appropriate amino acid alterations can be made on the basis of several criteria, including hydrophobicity, basic or acidic character, charge, polarity, size, the presence or absence of a functional group (e.g., —SH or a glycosylation site), and aromatic character. Assignment of various amino acids to similar groups based on the properties above will be readily apparent to the skilled artisan; further appropriate amino acid changes can also be found in Bowie et al. (Science 247:1306-1310(1990)). Other alterations of a TRX polypeptide of the invention include, for example, glycosylations, acetylations, carboxylations, phosphorylations, ubiquitination, labeling (e.g., with radionuclides), enzymatic modifications, incorporation of analogs of an amino acid, substituted linkages, and other modifications known in the art, both naturally and non-naturally occurring.

The invention described herein also relates to fragments of the isolated polypeptides described herein. The term “fragment” is intended to encompass a portion of a polypeptide described herein that retains one or more functions or biological activities of the isolated polypeptide, as described above, such as immunogenic or antigenic function (e.g., SEQ ID NO:8). For example, the fragment can be from at least 20 contiguous amino acids to at least 200 contiguous amino acids, more preferably at least 50 contiguous amino acids, even more preferably at least 100 contiguous amino acids, even more preferably at least 150 contiguous amino acids of a TRX polypeptide (e.g., SEQ ID NO:9-SEQ ID NO: 10).

A TRX polypeptide can also be a fusion protein comprising all or a portion of the TRX amino acid sequence fused to one or more additional components. Representative fusion partners include immunoglobulins, β-galactosidase, trpE, protein A, β-lactamase, αamylase, alcohol dehydrogenase, and yeast α-mating factor. Additional components, such as radioisotopes and antigenic tags, can be selected to assist in the isolation or purification of the polypeptide or to extend the half life of the polypeptide; for example, a hexahistidine tag would permit ready purification by nickel chromatography. Furthermore, polypeptides of the present invention can be progenitors of a TRX polypeptide. Progenitors are molecules that are cleaved to form an active TRX polypeptide.

A TRX polypeptide or peptide can be isolated from naturally-occurring sources, chemically synthesized, or produced by recombinant or cell-free systems. For example, a nucleic acid molecule described herein can be used to produce a recombinant form of the encoded polypeptide via microbial or eukaryotic cellular processes. Ligating the polynucleotide sequence into a gene construct, such as an expression vector, and transforming or transfecting into hosts, either eukaryotic (yeast, avian, insect, plant or mammalian) or prokaryotic (bacterial cells), are standard procedures used in producing other well known proteins. Similar procedures, or modifications thereof, can be employed to prepare recombinant polypeptides according to the present invention by microbial means, cell-free methods, or tissue-culture technology.

Antibodies

According to the invention, the measurement of TRX levels can be determined using one or more antibodies that bind to a TRX polypeptide or peptide sequence. For instance, polyclonal and monoclonal antibodies, including non-human and human antibodies, humanized antibodies, chimeric antibodies, and antigen-binding fragments thereof, which bind to a TRX polypeptides are within the scope of the invention (see, e.g., Current Protocols in Immunology, John Wiley & Sons, New York (1994); EP Application 173,494 (Morrison); International Patent Application WO86/01533 (Neuberger); and U.S. Pat. No. 5,225,539 (Winters)). A mammal, such as a chicken, mouse, rat, hamster, or rabbit, can be immunized with an immunogenic form of a TRX polypeptide (e.g., the protein or a peptide comprising an antigenic fragment of the protein which is capable of eliciting an antibody response). Techniques for conferring immunogenicity on a protein or peptide include conjugation to carriers or other techniques well known in the art. The protein or polypeptide can be administered in the presence of an adjuvant. The progress of immunization can be monitored by detection of antibody titers in plasma or serum. Standard ELISA or other immunoassays can be used with the immunogen as antigen to assess the levels of antibody.

Following immunization, anti-peptide antisera can be obtained, and if desired, polyclonal antibodies can be isolated from the serum. Monoclonal antibodies can also be produced by standard techniques that are well known in the art (Kohler and Milstein, Nature 256:495-497 (1975); Kozbar et al., Immunology Today 4:72 (1983); and Cole et al., Monoclonal Antibodies and Cancer Therapy, Alan R. Liss, Inc., pp. 77-96 (1985)). The term “antibody” as used herein is intended to include fragments thereof, such as Fab and F(ab)₂′. Such antibodies, in conjunction with a label, such as a fluorescent, enzymatic, or radioactive label, can be used to assay for the presence of the expressed TRX, e.g., a fluid or cell sample. Such antibodies can also be used in an immunoabsorption process, such as an ELISA, to isolate a TRX polypeptide. Biological samples which can be assayed include primate, particularly human, fluids, tissues, or cells e.g., whole blood, blood fractions, or blood cells. Examples include plasma, serum, and PBMC.

Detection of antibodies can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include GFP, luciferase, luciferin, and aequorin, and examples of suitable radioactive material include ¹²⁵I, ¹³¹I, ³⁵S or ³H.

The methods of the invention also encompass the use of commercially available antibodies for TRX. Commercially available antibodies include, but not limited to, chicken anti-thioredoxin polyclonal antibody, unconjugated (BioChain); mouse anti-thioredoxin monoclonal antibody, unconjugated, Clone TRX-01 (Novus Biologicals); mouse anti-human thioredoxin monoclonal antibody, unconjugated, Clone 2B1 (Serotec, Inc.); mouse anti-thioredoxin monoclonal antibody, unconjugated, Clone 2C9 (Stressgen Bioreagents); mouse anti-human thioredoxin monoclonal antibody, unconjugated, Clone 1.VB.2 and mouse anti-human thioredoxin monoclonal antibody, unconjugated, Clone 1.VB.2 (United States Biological); goat anti-thioredoxin polyclonal antibody, unconjugated, mouse anti-thioredoxin (full length, TRX 1-104) monoclonal antibody, FITC-labeled, Clone 2G11, mouse anti-thioredoxin (full length, TRX 1-104) monoclonal antibody, unconjugated, Clone 2G11, mouse anti-thioredoxin (truncated, TRX 1-84) monoclonal antibody, unconjugated, Clone 7D11 (TRX4), and mouse anti-thioredoxin (truncated, TRX 1-84) monoclonal antibody, unconjugated, Clone 4H9 (TRX2) (all available from BD Biosciences Pharmingen); rabbit anti-thioredoxin (THRx) polyclonal antibody, unconjugated and rabbit anti-thioredoxin (THRx) polyclonal antibody, unconjugated (Alpha Diagnostic International, Inc.).

Nucleic Acid-Based Diagnosis and Monitoring

The invention encompasses methods of monitoring and/or assisting in diagnosing TRX-related diseases by measuring TRX nucleic acid levels (e.g., transcript levels). The invention further encompasses methods of determining the biological activity of HDAC inhibitors (e.g., SAHA) or other therapeutic agents by measuring TRX nucleic acid levels (e.g., transcript levels). Because of the relationship between the TRX nucleic acid levels and HDAC inhibitors as determined herein, the disclosed methods can be used to monitor the biological activity of such inhibitors. A decrease in TRX nucleic acid levels upon administration of one or more therapeutic agents is indicative of biological activity of such agents. The continuing elevation of TRX nucleic acid levels upon administration of one or more therapeutic agents is indicative of a need to increase dosage, and thereby increase the biological activity of such agents. Thus, the disclosed methods can be used to infer the efficacy of the treatments with HDAC inhibitors (e.g., SAHA) and other therapeutic agents.

In the methods of the invention, a biological sample from an individual is used, such as an individual who is undergoing treatment with at least one HDAC inhibitor or other therapeutic agent. The biological sample can comprise whole blood, plasma, serum, PBMC, cerebrospinal fluid, urine, nasal secretion, saliva, cancer cells, tumor cells (e.g., biopsy sample), or any other bodily fluid, tissue, or cells as indicated herein. In a preferred embodiment, the biological sample is a serum or plasma sample. The biological sample is assessed for TRX transcript levels (e.g., mRNA transcript). In one embodiment of the invention, one or more TRX primers or probes described above can be used to detect the levels of TRX transcript. The TRX primers or probes are contacted with the biological sample from an individual. This results in a mixture of the TRX primers or probes and the biological sample. The mixture is maintained under appropriate conditions to allow hybridization to a TRX transcript (e.g., mRNA transcript). The levels of TRX transcript are then assessed by the appropriate method.

In other methods of the invention, a biological sample from an individual, such as an individual who is suspected of having a TRX-related disorder, is used. The biological sample can also be from an individual who is suspected of having a disorder, but who does not demonstrate symptoms thereof. The biological sample can comprise, for example, blood, serum, plasma, PBMC, cerebrospinal fluid, urine, nasal secretion, saliva, cancer cells, tumor cells (e.g., biopsy sample), or any other bodily fluid, tissue, or cells as indicated herein. The biological sample is assessed for levels of TRX transcript (e.g., mRNA transcript). In one embodiment of the invention, one or more of TRX probes or primers described above can be used to detect the levels of TRX transcript. The biological sample can contain more than one TRX transcript e or derivatives or fragments thereof. The biological sample is contacted with the primers or probes. The contacted sample is maintained under appropriate conditions to allow hybridization of the primers or probes to a TRX transcript. The levels of TRX transcript are then assessed.

The amount of TRX transcript can be determined by a variety of standard techniques, including RT-PCR (e.g., quantitative or real-time), Northern blots, dot blots, slot blots, microarray analysis, RNase protection analysis, etc. (see Ausubel, F. M. et al., eds., Current Protocols in Molecular Biology, John Wiley & Sons). In a preferred embodiment, the TRX transcript levels are determined by Northern blot analysis. In another preferred embodiment, the level of TRX transcript is determined by RT-PCR. For example, the amount of binding can be determined by using blood fractions, such serum, plasma, or PBMC. The blood fractions are incubated with TRX primers or probes, and then the amount of TRX can be determined. Typically, the amount of probe that binds to the TRX nucleic acid in the biological sample can be determined using a detector molecule attached directly or indirectly to the probe. As an alternative, the amount of RT-PCR product can be determined by gel analysis with ethidium bromide. In one aspect, the transcripts in the biological sample are separated on a gel and transferred to a solid support, the support is then incubated with labeled or unlabeled probes, and the amount of TRX is assessed by enzymatic, radiographic, or fluorescent signals. In another embodiment, the TRX probe is pre-attached to a solid support.

For assessing the biological activity of a therapeutic agent (e.g., HDAC inhibitor such as SAHA, etc.), the levels of TRX transcript in the biological sample are compared to a negative control. The term, “negative control,” as used in this embodiment, refers to an amount of TRX transcript that correlates with pre-treatment levels. A negative control can be determined for this embodiment by measuring TRX transcript levels in the individual prior to treatment, or by determining standard TRX transcript levels in untreated samples (e.g., an untreated cancer cell line). The levels of TRX transcript in a biological sample can also be compared to a positive control. For example, a “positive control” in this embodiment can be determined by measuring TRX transcript levels in an individual that is responsive to the same therapeutic agent used for the biological sample, or by determining standard TRX transcript levels in treatment-responsive samples (e.g., a cancer cell line) contacted with the therapeutic agent. The levels of TRX transcript in the biological sample can then be compared to the control levels. Where the TRX transcript levels in the biological sample are decreased relative to the untreated samples, the amount of decrease correlates with the biological activity of the therapeutic agent. Where the TRX transcript levels are equal to, or only slightly decreased relative to the untreated samples, this points to insufficient biological activity of the therapeutic agent. Insufficient biological activity may indicate the need for increased dosages of the therapeutic agent, while lack of apparent biological activity may indicate the need for an alternate treatment strategy.

For monitoring and/or assisting with diagnosis of a TRX-related disorder (e.g., cellular proliferation, inflammatory disease, autoimmune disease, liver disease, viral disease, or coronary disorder, etc.), the amount of TRX transcript in the biological sample, is compared to a reference amount. The term, “reference amount,” as used in this embodiment, refers to a level of TRX transcript that correlates with a diagnosis of a TRX-related disorder. A reference amount can be determined, for example, by comparing amounts of TRX transcript in biological samples from individuals known to have a TRX-related disorder (e.g., a “positive control sample”), with amounts of TRX transcript in a biological sample from individuals known not to have a TRX-related disorder (e.g., a “negative control sample”), and determining what amount of transcript correlates with disease. The reference amount can be assessed by determining the amounts of TRX transcript in positive and/or negative control samples, as indicated in this embodiment, concurrently with determining the amount of TRX transcript in the biological sample. Alternatively, the reference amount can be an amount determined from a biological sample from the same patient at an earlier time point (i.e., to determine onset, progression, regression, or stabilization of the disease or disorder). Where the TRX transcript levels in the biological sample are increased relative to the negative control samples (and, especially, where levels approach or exceed the levels in the positive control samples), this correlates with the onset or progression of a TRX-related disease or disorder. Where the TRX transcript levels are equivalent or nearly equivalent to the negative control samples, this indicates the absence, regression or stabilization TRX-related disease or disorder.

The present invention also includes kits to be used in methods of the invention. Kits can include the following components: (a) one or more TRX probes or primers; and, optionally; (b) labeling for the probes or primers. The kits can also include positive and negative controls as described herein, and instructions for use. The label can comprise a detectable agent, such as an enzyme, radioactive molecule, or fluorescent agent as indicated above. If the detectable agent is an enzyme that reacts with an added substrate to yield a colored product, such as horseradish peroxidase, the kit can also include the corresponding substrate. The TRX probe(s) in the kit can be adhered to a solid support.

Antibody-Based Diagnosis and Monitoring

The invention further encompasses methods for monitoring and/or assisting with diagnosis of TRX-related diseases by measuring TRX polypeptide levels. The invention encompasses methods of determining the biological activity of HDAC inhibitors (e.g., SAHA) or other therapeutic agents by measuring TRX polypeptide levels. Because of the relationship between the TRX levels and HDAC inhibitors as determined herein, the disclosed methods can be used to monitor treatment efficacy for such inhibitors. A decrease in TRX polypeptide levels upon administration of one or more therapeutic agents is indicative of biological activity of such agents. The continuing elevation of TRX polypeptide levels upon administration of one or more therapeutic agents is indicative of a need to increase dosage, and thereby increase the biological activity of such agents. Thus, the disclosed methods can be used to infer the efficacy of the treatments with HDAC inhibitors (e.g., SAHA) and other therapeutic agents.

In the methods of the invention, a biological sample from an individual is used, such as an individual who is undergoing treatment with at least one HDAC inhibitor or other therapeutic agent. The biological sample can comprise whole blood, plasma, serum, PBMC, cerebrospinal fluid, urine, nasal secretion, saliva, cancer cells, tumor sample (e.g., biopsy), or any other bodily fluid, tissue, or cells as indicated herein. In a preferred embodiment, the biological sample is a serum or plasma sample. The biological sample is assessed for TRX levels. In one embodiment of the invention, one or more TRX antibodies described above can be used to detect the levels of TRX polypeptide. The TRX antibody is contacted with the biological sample from an individual. This results in a mixture of the TRX antibody and the biological sample. The mixture is maintained under appropriate conditions to allow binding of antibody to a TRX polypeptide. The levels of TRX polypeptide are then assessed.

In other methods of the invention, a biological sample from an individual, such as an individual who is suspected of having a TRX-related disorder, is used. The biological sample can also be from an individual who is suspected of having a disorder, but who does not demonstrate symptoms thereof. The biological sample can comprise, for example, blood, serum, plasma, PBMC, cerebrospinal fluid, urine, nasal secretion, saliva, or any other bodily fluid, tissue, or cells as indicated herein. The biological sample is assessed for levels of TRX polypeptide. In one embodiment of the invention, one or more of TRX antibodies described above can be used to detect the levels of TRX polypeptide. The biological sample can contain more than one TRX polypeptide or active derivatives or fragments thereof. The biological sample is contacted with the anti-TRX antibody. The contacted sample is maintained under appropriate conditions to allow binding of antibody to a TRX polypeptide. The levels of TRX polypeptide are then assessed.

The amount of TRX polypeptide can be determined by a variety of standard techniques, including enzyme-linked immunosorbent assays (ELISAs) or other solid phase immunoassays, radioimmunoassay, nephelometry, electrophoresis, immunofluorescence (direct or indirect), immunohistochemistry, Western blots or other immunoblots, dot blots, slot blots, microarrays, etc. (see Ausubel, F. M. et al., eds., Current Protocols in Molecular Biology, John Wiley & Sons, including supplements through 1997, especially units 11.2 (ELISA) and 11.16 (Determination of Specific Antibody Titer)). In a preferred embodiment, the titer is determined by ELISA; in another preferred embodiment, the level of antibody binding is determined by Western blot. For example, the amount of binding can be determined by using blood fractions, such serum, plasma, or PBMC. The blood fractions are incubated with antibodies or antibody fragments, and then the amount of TRX polypeptide can be determined. Typically, the amount of antibody that binds to the TRX polypeptide in the biological sample can be determined using a detector antibody that binds to the anti-TRX antibody. In one aspect, the proteins in the biological sample are separated on a gel and transferred to a solid support, the support is then incubated with the antibodies or antibody fragments, and the amount of TRX is assessed by indirect immunofluorescence. In a particularly preferred embodiment, the TRX antibody or antibody fragment is pre-attached to a solid support.

For assessing the biological activity of a therapeutic agent (e.g., HDAC inhibitor such as SAHA, etc.), the levels of TRX polypeptide in the biological sample are compared to a negative control. The term, “negative control,” as used in this embodiment, refers to an amount of TRX polypeptide that correlates with pre-treatment levels. A negative control can be determined, for example, by measuring TRX polypeptide levels in the individual prior to treatment, or by determining standard TRX polypeptide levels in untreated cancer samples (e.g., a cancer cell line). The levels of TRX polypeptide in a biological sample can also be compared to a positive control. For example, a “positive control” as used in this embodiment can be determined by measuring TRX polypeptide levels in an individual that is responsive to the same therapeutic agent used in the biological sample, or by determining standard TRX polypeptide levels in treatment-responsive samples (e.g., a cancer cell line) contacted with this inhibitor. The levels of TRX polypeptide in the biological sample can then be compared to the control levels. Where the TRX polypeptide levels in the biological sample are decreased relative to the untreated samples, the amount of decrease correlates with the biological activity of the therapeutic agent. Where the TRX polypeptide levels are equal to, or only slightly decreased relative to the untreated samples, this points to insufficient biological activity of the therapeutic agent. Insufficient biological activity may indicate the need for increased dosages of the therapeutic agent, while lack of detectable biological activity may indicate the need for an alternate treatment strategy.

For diagnosing and/or monitoring a TRX-related disorder (e.g., cellular proliferation, inflammatory disease, autoimmune disease, liver disease, viral disease, or coronary disorder, etc.), the amount of TRX polypeptide in the biological sample, is compared to a reference amount. The term, “reference amount,” as used in this embodiment, refers to an amount TRX polypeptide that correlates with a diagnosis of a TRX-related disorder. A reference amount can be determined, for example, by comparing amounts of TRX polypeptide in biological samples from individuals known to have a TRX-related disorder (e.g., a “positive control sample”), with amounts of TRX polypeptide in a biological sample from individuals known not to have a TRX-related disorder (e.g., a “negative control sample” from a healthy individual), and determining what amount of polypeptide correlates with disease. The reference amount can be determined by determining the amounts of TRX polypeptide in positive and/or negative control samples, as indicated in this embodiment, concurrently with determining the amount of TRX polypeptide in the biological sample. Alternatively, the reference amount can be an amount determined from the same patient at an earlier time point (i.e., to determine progression and/or regression of the disease or disorder). Where the TRX polypeptide levels in the biological sample are increased relative to the negative control samples (and, particularly, when the levels approach or exceed the levels in the positive control samples), this correlates with the onset or progression of a TRX-related disease or disorder. Where the TRX polypeptide levels are equivalent or nearly equivalent to the negative control samples, this indicates the absence, regression or stabilization TRX-related disease or disorder.

The present invention also includes kits to be used in methods of the invention. Kits can include the following components: (a) an anti-TRX antibody or antibody fragment; and, optionally; (b) labeled detector antibody that binds to the antibody. The kits can also include positive and negative controls as described herein, and instructions for use. The detector antibody can comprise an antibody bound to a detectable agent, such as an enzyme, radioactive molecule, or fluorescent agent as indicated above. If the detector antibody is bound to an enzyme that reacts with an added substrate to yield a colored product, such as horseradish peroxidase, the kit can also include the corresponding substrate. The anti-TRX antibody or antibody fragment in the kit can be adhered to a solid support.

### EXAMPLES

The examples are presented in order to more fully illustrate the preferred embodiments of the invention. These examples should in no way be construed as limiting the scope of the invention, as defined by the appended claims.

### Example 1

### Effects of SAHA on Normal and Tumor Cells

Cell Culture

HeLa (human cervical carcinoma), WI38 (human lung fibroblast), WI38-VA13 (SV-40 transformed human lung fibroblast), MCF-7 (human breast adenocarcinoma), T-24 human bladder transitional cell carcinoma), and LNCAP (human prostate adenocarcinoma) were obtained from the American type culture collection and cultured in accordance with the instructions. ARP-1 (human multiple myeloma) was generously provided by Dr. J. Hardoc (Arkansas Cancer Research Center, Little Rock) and cultured as indicated by the source. SAHA was synthesized as described (Richon, V. M., et al., 1996, Proc. Natl. Acad. Sci. USA. 93:5705-5708), and was dissolved and diluted in DMSO.

Cell Growth and Viability

Cells were plated in dishes varying in size from 24-well plates to 15 cm² dishes and treated 18-24 hrs after plating with the indicated drug concentration of SAHA. Cells were harvested by trypsinization. Cell number and viability were measured by trypan blue dye exclusion and by Guava PCA-96 via count flex reagent according to the manufacturer's instruction (Guava).

RNA Isolation and Northern Blotting

Cells were cultured and recovered by centrifugation and RNA was prepared as described (Richon, V. M., et al., 1996, Proc. Natl. Acad. Sci. USA. 93:5705-5708). Total RNA (10 μg) was analyzed by northern blotting using a ³²P-labeled 1.1-kb TBP-2 coding region cDNA probe or a 500 bp cDNA probe for human TRX (Butler et al., 2002, Proc. Natl. Acad. Sci. USA 99:11700-11705). Loading was determined using a ³²P labeled 50-mer 18s rRNA oligonucleotide.

Western Blot and Immunoprecipitation

Typically, whole cell lysates were prepared using a buffer (50 mM Tris-HCL pH 8, 300 mM NaCl, 5 mM EDTA, 0.5% NP-40, inhibitor pellet from Roche). Next, 10-25 μg of cell extract were subjected to standard SDS-poly acrylamide gel electrophoresis using 10%, 15%, and 18% criterion gels (Bio-Rad). After transferring to PVDF membrane (Amersham Biosciences), the membranes were blocked with 5% BSA PBS-Tween (0.1%). The membranes were then incubated with specific primary antibody and horseradish peroxidase-labeled secondary antibody. Immunoprecipitation was based on the protocol of Santa Cruz Biotechnology. Briefly, 100-500 μg of total cellular protein were applied and incubated with protein agarose (Ausubel, F. M., et al. Current Protocols in Molecular Biology. 10.16 John Wiley & Sons. 2001).

Antibodies

TBP-2 antiserum was generated in rabbits (Pocono Farms) using following sequence (peptide) CYM DVI PED HRL ESP (SEQ ID NO:7). Anti-TRX was prepared in the laboratory of Dr. Arne Holmgren (Karolinska institute) as a goat polyclonal antibody. Anti-tubulin was purchased from Oncogene.

Results

TBP-2 has been identified as a protein that associates with the active (reduced) form of TRX, a dithiol-reducing redox regulatory protein (Nishiyama, A., et al., 1999, J. Biol. Chem. 274, 21645-21650). Binding of TBP-2 to TRX both inhibits the thiol-reducing activity and reduces the level of expression of TRX. To study the effects of SAHA treatment on normal and tumor cells, studies were performed on the normal human fibroblast cell line WI38, and its SV40 transformed counterpart, the tumor cell line VA13. A specific monoclonal antibody to TRX (clone 2G11) produced in mouse, and a polyclonal antibody to full length TRX as a capture antibody, produced in goat, was used.

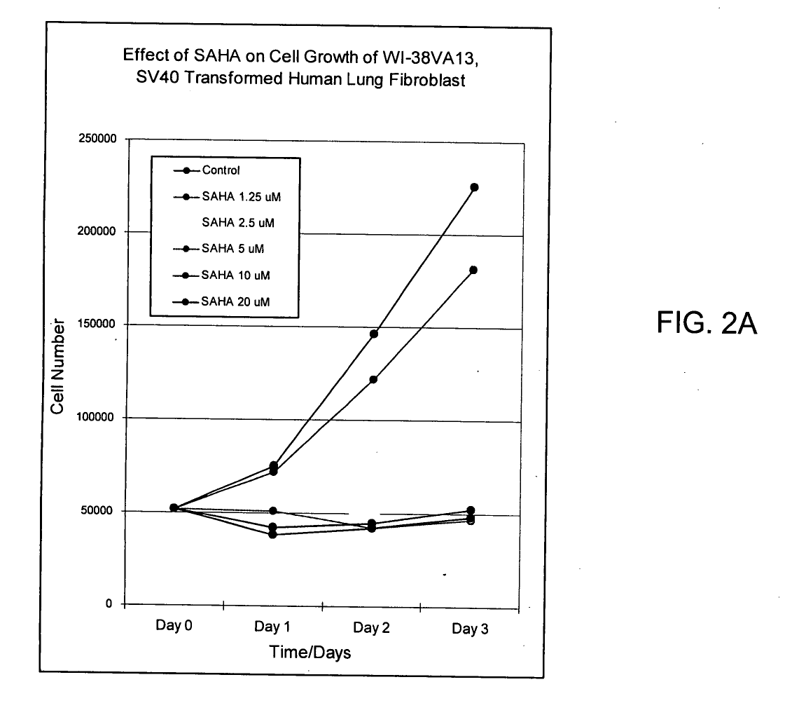

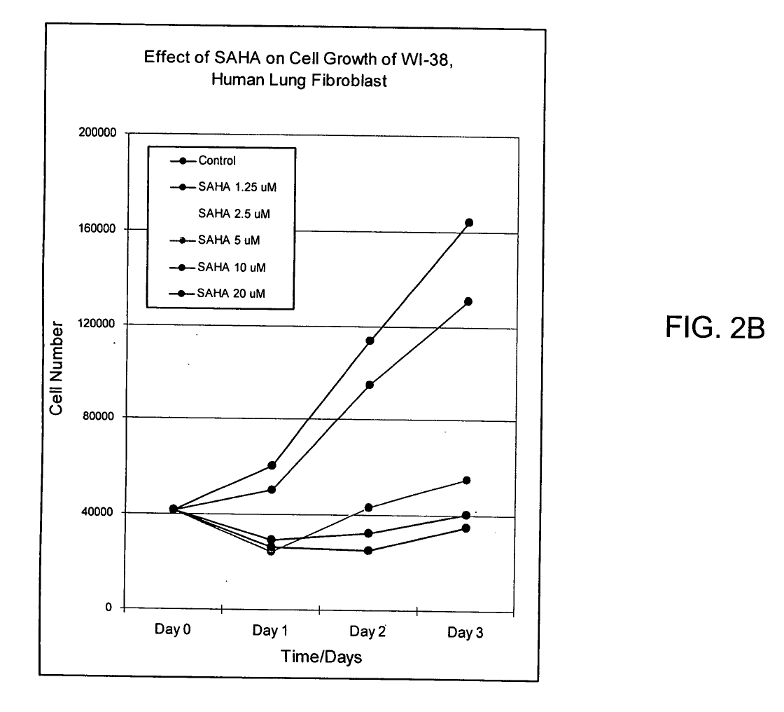

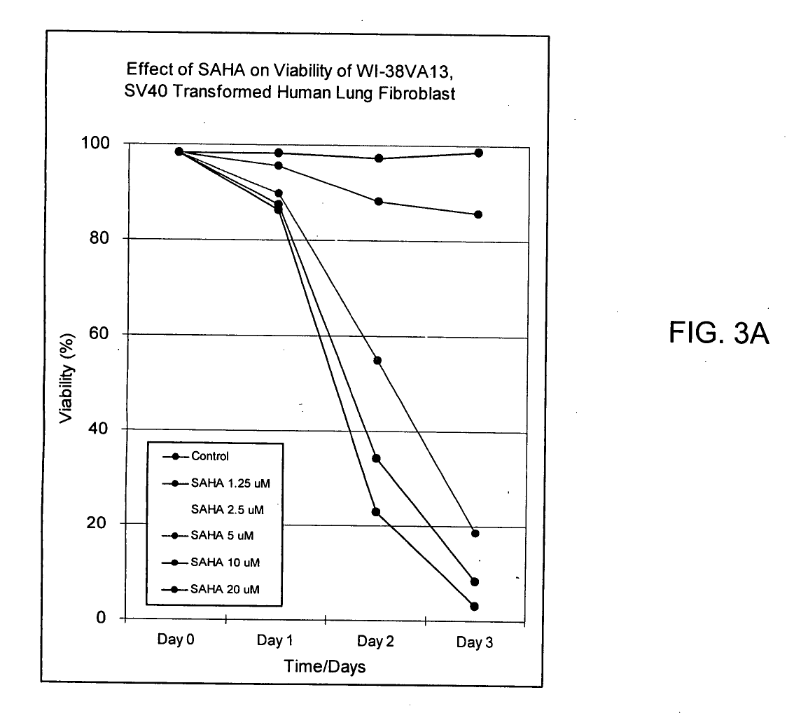

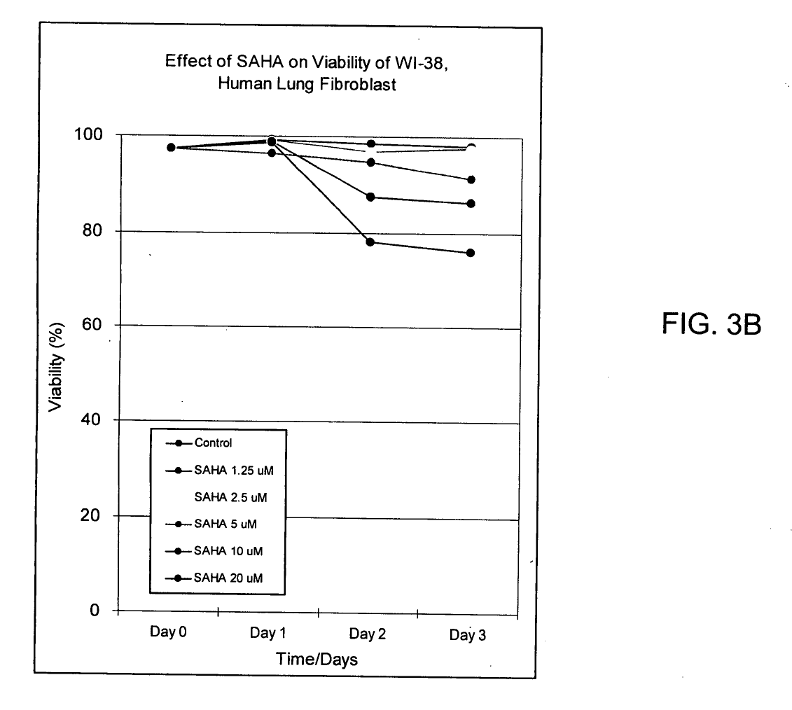

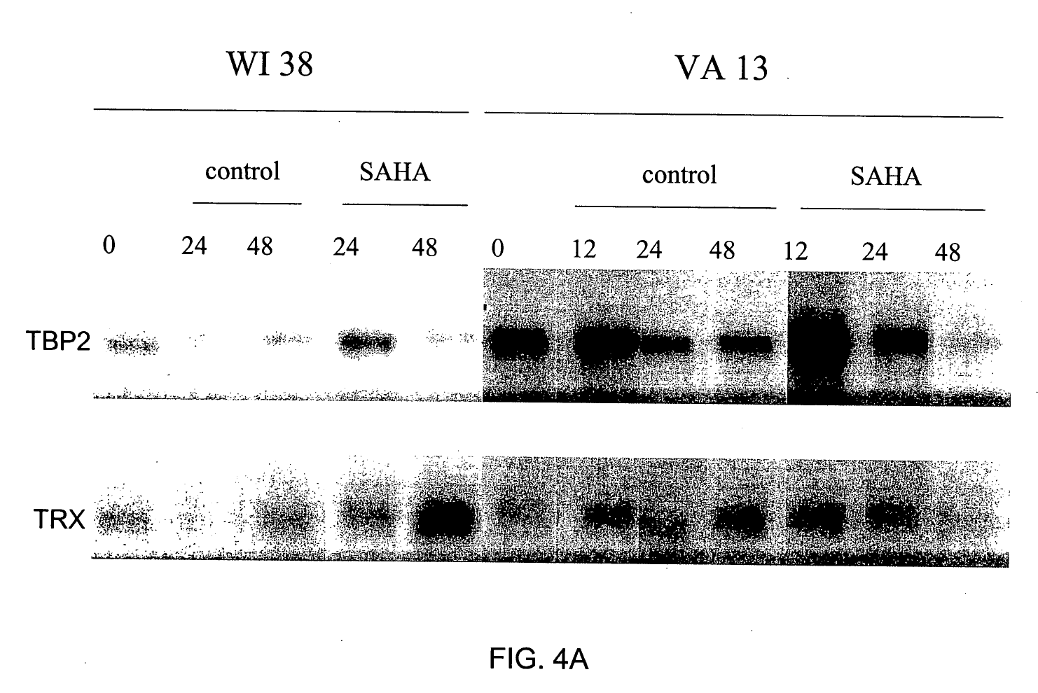

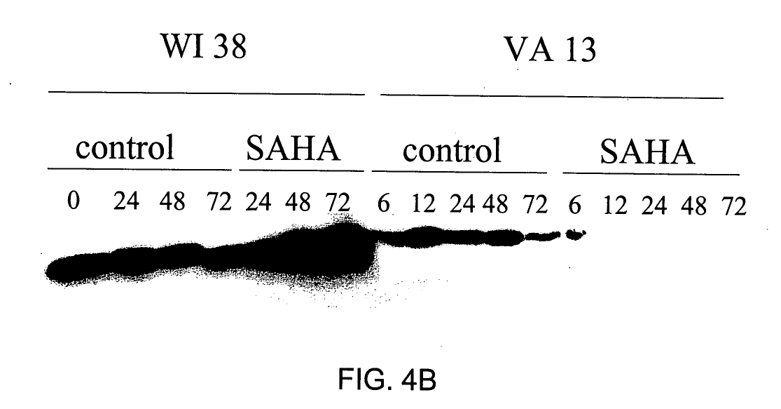

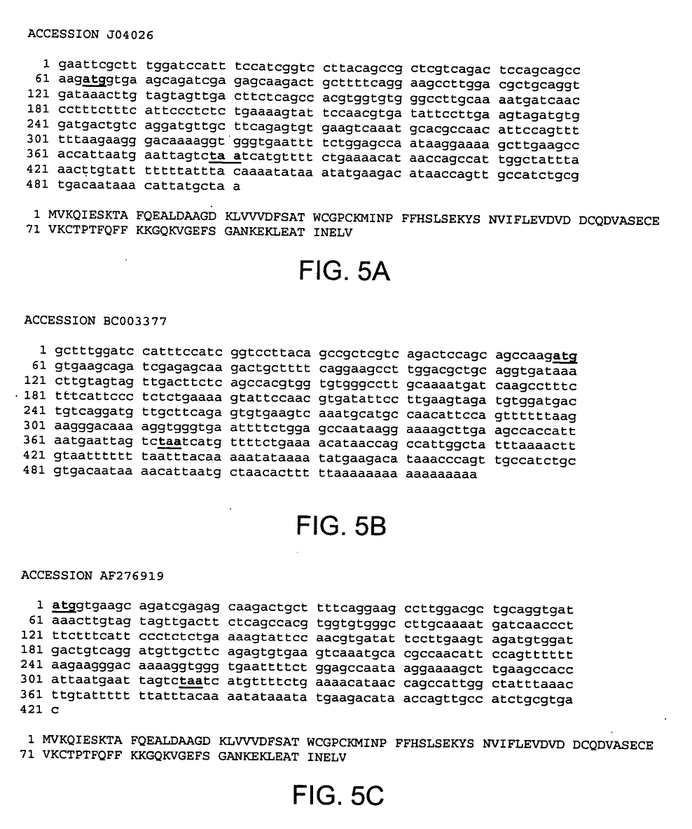

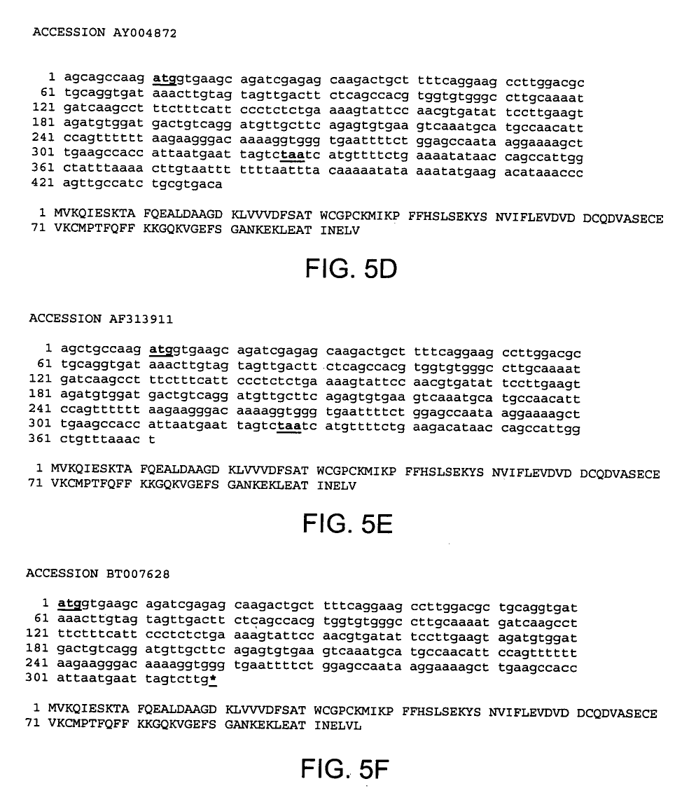

The results indicated that SAHA induced growth arrest and apoptosis in the transformed VA13, whereas little or no apoptosis was seen in the normal fibroblast WI38 cells (FIGS. 2-3). We hypothesized that the mechanism behind the resistance of normal cells to SAHA was related to their response to oxidative stress. We then focused on TRX, a major intracellular redox regulatory protein. TRX mRNA expression as well as protein levels in cultured normal fibroblasts WI38 increased significantly upon SAHA treatment (FIGS. 4A-4B). In the transformed VA13 fibroblasts, TRX mRNA expression was unchanged and protein levels quickly decrease to undetectable levels (FIGS. 4A-4B). These results indicate that SAHA treatment appears to abrogate the ability of tumor cells, but not of normal cells, to upregulate TRX expression, and that SAHA induced downregulation of TRX leads to tumor cell apoptosis.

### Example 2

### ELISA Method for Measuring Plasma or Serum TRX

To measure plasma or serum TRX, a protocol is adapted from Pekkari et al. (JBC 275(48); 37474-37480, 2000). Patient plasma or serum samples (e.g., 10 ml) are aliquotted and frozen until analyzed. Specific monoclonal mouse anti-TRX (clone 2G11), polyclonal goat anti-TRX and purified TRX protein is provided by Dr. Holmgren, Karolinska Institutet. Standard samples of purified TRX are kept in aliquots of 100 μg/ml in PBS with 0.5% bovine serum albumin and kept at −70° C. Each aliquot is discarded after being thawed once.

96-well plates are coated with 50 μl of 10 μg/ml anti-TRX antibodies in PBS. The plates are incubated at 4° C. overnight. The coating mixture is discarded, and 200 μl of incubation buffer (0.5% bovine serum albumin, 0.05% Tween 20, 0.02% NaN₃ in PBS) is added. This mixture is incubated for 2 hours at room temperature to block unspecific protein binding sites. The plates are washed four times with washing buffer (0.05% Tween 20 in 0.9% saline). Standard dilutions are made for TRX and samples in incubation buffer. Fifty microliters of standard or sample is added to the wells in duplicate. The plates are incubated at 4° C. overnight. The plates are washed four times with washing buffer.

Fifty microliters of biotinylated goat anti-TRX polyclonal antibody (at 2 μg/ml) is added. The plates are incubated 2 hours at room temperature. The plates are washed four times with washing buffer. Fifty microliters of alkaline phosphatase conjugated streptavidin diluted 1:1000 in incubation buffer is added. The plates are incubated 1 hour at room temperature. The plates are washed six times in washing buffer. Fifty microliters per well of 1 mg/ml p-nitrophenyl phosphate is dissolved in 10% diethanolamine, 0.02% NaN₃, 0.5 mM MgCl₂, pH 9.8. The absorbance is measured at 405 nm in a microplate reader.

It is important to measure hemoglobin levels in the samples, as erythrocytes contain high amounts of TkX. Thus, hemolysis can significantly contribute to increased plasma or serum levels of TRX. A commercially available test for hemoglobin is used to determine the amount of erythrocyte hemolysis, and correct for the contamination of the plasma TRX by the erythrocyte TRX release (Nakamura H, Int Immunol 8(4); 603-611, 1996). The contribution of released intracellular full length TRX due to hemolysis, compared to the total determined extracellular TRX determined by ELISA, is calculated according to Nakamura et al (Nakamura H, Int Immunol 8(4); 603-611, 1996).

The details of one or more embodiments of the invention have been set forth in the accompanying description above. Although any methods and materials similar or equivalent to those described herein can be used in the practice or testing of the present invention, the preferred methods and materials are now described. Other features, objects, and advantages of the invention will be apparent from the description and from the claims.

In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. Unless expressly stated otherwise, the techniques employed or contemplated herein are standard methodologies well known to one of ordinary skill in the art.

All patents and publications cited in this specification are hereby incorporated by reference herein in their entirety, including U.S. Pat. No. 5,668,179 issued Sep. 16, 1997, U.S. Pat. No. 5,773,474 issued Jun. 30, 1998, U.S. Pat. No. 5,055,608 issued Oct. 8, 1991, U.S. Pat. No. 5,175,191 issued Dec. 29, 1992, U.S. Pat. No. 5,330,744 issued Jul. 19, 1994, U.S. Pat. No. 5,608,108 issued Mar. 4, 1997, U.S. Pat. No. 5,840,960 issued Nov. 24, 1998, U.S. Pat. No. 5,932,616 issued Aug. 3, 1999, U.S. Pat. No. 6,087,367 issued Jul. 11, 2000, U.S. Pat. No. 6,511,990 issued Jan. 28, 2003, U.S. Pat. Reissue No. 38,506 issued Apr. 20, 2004, U.S. patent application Ser. No. 10/095,109 filed Mar. 8, 2002, U.S. patent application Ser. No. 10/273,401 filed Oct. 16, 2002, U.S. patent application Ser. No. 10/281,875 filed Oct. 25, 2002, U.S. application Ser. No. 10/413,422 filed Apr. 15, 2003, U.S. application Ser. No. 10/817,688 filed Apr. 1, 2004, and in particular, U.S. patent application Ser. No. 10/369,094 filed Feb. 14, 2003, and U.S. Provisional Application Ser. No. 60/357,383 filed Feb. 15, 2002.

### REFERENCES

Jenuwein T, Allis C D: Translating the histone code. Science 2001; 293(5532): 1074-80.
Spotswood H T, Turner B M: An increasingly complex code. J Clin Invest 2002; 110(5): 577-82.
Zhang Y, Reinberg D: Transcription regulation by histone methylation: interplay between different covalent modifications of the core histone tails. Genes Dev 2001; 15(18): 2343-60.
Agalioti T, Chen G, Thanos D: Deciphering the transcriptional histone acetylation code for a human gene. Cell 2002; 111(3): 381-92.
Richards E J, Elgin S C: Epigenetic codes for heterochromatin formation and silencing: rounding up the usual suspects. Cell 2002; 108(4): 489-500.
Timmermann S, Lehrmann H, Polesskaya A, Harel-Bellan A: Histone acetylation and disease. Cell Mol Life Sci 2001; 58(5-6): 728-36.
Wang C, Fu M, Mani S, Wadler S, Senderowicz A M, Pestell R G: Histone acetylation and the cell-cycle in cancer. Front Biosci 2001; 6:D610-29.
Marks P, Rifkind R A, Richon V M, Breslow R, Miller T, Kelly W K: Histone deacetylases and cancer: causes and therapies. Nat Rev Cancer 2001; 1(3): 194-202.
Jones P A, Baylin S B: The fundamental role of epigenetic events in cancer. Nat Rev Genet 2002; 3(6): 415-28.
Marks P, Miller T, Richon V: Histone Deacetylases. Current Opinion in Pharmacology 2003, 3:344-351.
Grozinger C M, Schreiber S L: Deacetylase enzymes: biological functions and the use of small-molecule inhibitors. Chem Biol 2002; 9(1): 3-16.
Johnstone R W, Licht J D: Histone deacetylase inhibitors in cancer therapy: is transcription the primary target? Cancer Cell 2003; 4(1): 13-8.
de Ruijter A J, van Gennip A H, Caron H N, Kemp S, van Kuilenburg A B: Histone deacetylases (HDACs): characterization of the classical HDAC family. Biochem J 2003; 370(Pt 3):737-49.
Khochbin S, Verdel A, Lernercier C, Seigneurin-Bemy D: Functional significance of histone deacetylase diversity. Curr Opin Genet Dev 2001; 11(2): 162-6.
Verdin E: Proceedings Novartis Foundation Symposium 2003; 259.
Cress W D, Seto E: Histone deacetylases, transcriptional control, and cancer. J Cell Physiol 2000; 184(1):1-16.
McKinsey T A, Zhang C L, Olson E N: Control of muscle development by dueling HATs and HDACs. Curr Opin Genet Dev2001; 11(5):497-504.
Umov F D, Yee J, Sachs L, Collingwood T N, Bauer A, Beug H, Shi Y B, Wolffe A P: Targeting of N-CoR and histone deacetylase 3 by the oncoprotein v-erbA yields a chromatin infrastructure-dependent transcriptional repression pathway. EMBO J. 2000; 19(15):4074-90.
Yoshida M, Kijima M, Akita M, Beppu T: Potent and specific inhibition of mammalian histone deacetylase both in vivo and in vitro by trichostatin A. J Biol Chem 1990; 265(28):17174-9.
Richon V M, Emiliani S, Verdin E, Webb Y, Breslow R, Rifkind R A, Marks P A: A class of hybrid polar inducers of transformed cell differentiation inhibits histone deacetylases. Proc Natl Acad Sci USA 1998; 95(6):3003-7.
Furumai R, Matsuyama A, Kobashi N, Lee K H, Nishiyama M, Nakajima H, Tanaka A, Komatsu Y, Nishino N, Yoshida M, Horinouchi S: FK228 (depsipeptide) as a natural prodrug that inhibits class I histone deacetylases. Cancer Res 2002; 62(17):4916-21.
Singh S B, Zink D L, Liesch J M, Mosley R T, Dombrowski A W, Bills G F, Darkin-Rattray S J, Schmatz D M, Goetz M A: Structure and chemistry of apicidins, a class of novel cyclic tetrapeptides without a terminal alpha-keto epoxide as inhibitors of histone deacetylase with potent antiprotozoal activities. J Org Chem 2002; 67(3):815-25.
Furumai R, Komatsu Y, Nishino N, Khochbin S, Yoshida M, Horinouchi S: Potent histone deacetylase inhibitors built from trichostatin A and cyclic tetrapeptide antibiotics including trapoxin. Proc Natl Acad Sci USA 2001; 98(1):87-92.
Phiel C J, Zhang F, Huang E Y, Guenther M G, Lazar M A, Klein P S: Histone deacetylase is a direct target of valproic acid, a potent anticonvulsant, mood stabilizer, and teratogen. J Biol Chem 2001; 276(39): 36734-41.
Boivin A J, Momparler L F, Hurtubise A, Momparler R L: Anti-neoplastic action of 5-aza-2′-deoxycytidine and phenylbutyrate on human lung carcinoma cells. Anticancer Drugs 2002; 13(8): 869-74.
Prakash S, Foster B J, Meyer M, Wozniak A, Heilbrun L K, Flaherty L, Zalupski M, Radulovic L, Valdivieso M, LoRusso P M: Chronic oral administration of CI-994: a phase 1 study. Invest New Drugs 2001; 19(1): 1-11.
Saito A, Yamashita T, Mariko Y, Nosaka Y, Tsuchiya K, Ando T, Suzuki T, Tsuruo T, Nakanishi O: A synthetic inhibitor of histone deacetylase, MS-27-275, with marked in vivo antitumor activity against human tumors. Proc Natl Acad Sci USA 1999; 96(8):4592-7.
Frey R R, Wada C K, Garland R B, Curtin M L, Michaelides M R, Li J, Pease L J, Glaser K B, Marcotte P A, Bouska J J, Murphy S S, Davidsen S K: Trifluoromethyl ketones as inhibitors of histone deacetylase. Bioorg. Med. Chem. Lett. 2002; 12(23):3443-7.
Finnin M S, Donigian J R, Cohen A, Richon V M, Rifkind R A, Marks P A, Breslow R, Pavletich N P: Structures of a histone deacetylase homologue bound to the TSA and SAHA inhibitors. Nature 1999; 401(6749):188-93.
Butler L M, Zhou X, Xu W-S, Scher H I, Rifkind R A, Marks P A, Richon V M: The histone deacetylase inhibitor SAHA arrests cancer cell growth, up-regulates thioredoxin-binding protein-2, and down-regulates thioredoxin. Proc Natl Acad Sci USA 2002; 99(18): 11700-11705.
Glaser K B, Staver M J, Waring J F, Stender J, Ulrich R G, Davidsen S K: Gene Expression Profiling of Multiple Historie Deacetylase (HDAC) Inhibitors: Defining a Common Gene Set Produced by HDAC Inhibition in T24 and MDA Carcinoma Cell Lines. Mol Cancer Ther 2003; 2(2):151-63.
Van Lint C, Emiliani S, Verdin E: The expression of a small fraction of cellular gene is changed in response to histone hyperacetylation. Gene Expr. 1996; 5:245-254.
Rascle A, Johnston J A, Amati B: Deacetylase activity is required for recruitment of the basal transcription machinery and transactivation by STAT5. Mol Cell Biol 2003; 23(12):4162-73.
Sambucetti L C, Fischer D D, Zabludoff S, Kwon P O, Chamberlin H, Trogani N, Xu H, Cohen D: Histone deacetylase inhibition selectively alters the activity and expression of cell cycle proteins leading to specific chromatin acetylation and antiproliferative effects. J Biol Chem 1999; 274(49): 34940-7.
Richon V M, Sandhoff T W, Rifkind R A, Marks P A: Histone deacetylase inhibitor selectively induces p21WAF1 expression and gene-associated histone acetylation. Proc Natl Acad Sci USA 2000; 97(18): 10014-9.
Richon V. M., Webb Y., Merger R., Sheppard T., Jursic B., Ngo L., Civoli F., Breslow R., Rifkind R. A., Marks P. A. Second generation hybrid polar compounds are potent inducers of transformed cell differentiation. Proc. Natl. Acad. Sci. USA, 93: 5705-5708, 1996.
Grunstein M. Histone acetylation and chromatin structure and transcription. Nature (Lond.), 389: 349-352, 1997.
Qui L., Kelson M. J., Hanse C., West M. L., Fairlie D. P., Parsons P. G. Anti-tumor activity in vitro and in vivo of selective differentiating agents containing hydroxamide. Br. J. Cancer, 80: 1252-1258, 1990.
He, L.-Z., Tolentino, T., Grayson, P., Zhong, S., Warrell, R. P. Jr., Rifkind, R. A., Marks, P. A., Richon, V. M., Pandolfi, P. P. 2001. Histone deacetylase inhibitors induce remission in transgenic models of therapy-resistant acute promyelocytic leukemia. J. Clin. Invest. 108: 1321-1330.
Kelly W. K., O'Connor O. A. and Marks P. A., 2002, Histone deacetylase inhibitors: from target to clinical trials. Expert Opin. Investig. Drugs 11; 1695-1713.
Kelly, Wm. K., Richon, V. M., O'Connor, O., Curley, T., MacGregor-Curtelli, B., Tong, W., Klang, M., Schwartz, L., Richardson, S., Rosa, E., Drobnjak, M., Cordon-Cordo, C., Chiao, J. H., Rifkind, R., Marks, P. A., Scher, H. 2003. Phase I Clinical Trial of Histone Deacetylase Inhibitor: Suberoylanilide Hydroxamic Acid Administered Intravenously. Clin Cancer Res 9: 3578-3588.
Arner, E. S. and Holmgren, A. 2000. Physiological functions of thioredoxin and thioredoxin reductase. Eur. J. Biochem. 267: 6102-6109.
Powis G, Mustacich D, Coon A, 2000, The role of the redox protein thioredoxin in cell growth and cancer, Free Radical Biology & Medicine 29; 312-322
Saitoh, M., Nishitoh, H., Fujii, M., Takeda, K., Tobiume, K, Sawada, Y., Kawabata, M., Miyazono, K., and Ichijo, H. 1998 Mammalian thioredoxin is a direct inhibitor of apoptosis signal-regulating kinase (ASK) 1. EMBO J. 17, 2596-2606.
Yokomizo A., Ono M., Manri H., Makino Y., Ohga T., Mada M., Okamoto T., Yodoi J., Kuwano M., Kohno K. Cellular levels of thioredoxin associated with drug sensitivity to cisplatin, mitomycin C, doxorubicin, and etoposide. Cancer Res., 55: 4293-4296, 1995.
Kakolyris, S., Giatromanolaki, A., Koukourakis, M., Powis, G., Souglakos, J., Sivridis, E., Georgoulias, V., Gatter, K. C., Harris, A. L. 2001. Thioredoxin Expression Is Associated with Lymph Node Status and Prognosis in Early Operable Non-Small Cell Lung Cancer. Clin Cancer Res 7: 3087-3091.
Soini Y, Kahlos K, Napankangas U, Kaarteenaho-Wiik R, Säily M, Koistinen P, Paakko P, Holmgren A, Kinnula V L 2001. Widespread expression of thioredoxin and thioredoxin reductase in non-small cell lung carcinoma. Clin Cancer Res 7:1750-1757.
Pekkari, K., Ramanathan, G., Amer, E. S. J., and Holmgren, A. 2000. Truncated thioredoxin is a mitogenic cytokine for resting human peripheral blood mononuclear cells and is present in human plasma. J. Biol. Chem, 275, 37474-37480
Nakamura, H., De Rosa, S., Roederer, M., Anderson, M. T., Dubs, J. G., Yodoi, J., Holmgren, A., & Herzenberg, L. A. 1996. Elevation of plasma thioredoxin levels in HIV-infected individuals. Int. Immunol. 8, 603611.
Kamsler A, Daily D, Hochman A, Stern N, Shiloh Y, Rotman G, Barzilai A, 2001, Increased oxidative stress in ataxia telangiectasia evidenced by alterations of redox state of brains from Atm-deficient mice. Cancer Res. 61:1849-1854.

1. A method for monitoring dosage of a histone deacetylase inhibitor for treatment of cellular hyperproliferation, comprising:
(a) measuring levels of thioredoxin transcript in a biological sample from a patient before treatment;
(b) measuring levels of thioredoxin transcript in a biological sample from the patient during treatment;
(c) comparing the levels of the thioredoxin transcript in (a) and (b), such that continuing elevation of levels of the transcript during treatment indicates insufficient dosage of the histone deacetylase inhibitor.

2. The method of claim 1, wherein the histone deacetylase inhibitor is selected from the group consisting of hydroxamates, cyclic peptides, aliphatic acids, benzamides, and electrophilic ketones.

3. The method of claim 2, wherein the histone deacetylase inhibitor is selected from the group consisting of suberoylanilide hydroxamic acid, pyroxamide, CBHA, trichostatin A, trichostatin C, salicylihydroxamic acid, azelaic bishydroxamic acid, azelaic-1-hydroxamate-9-anilid-e, 6-(3-chlorophenylureido)carpoic hydroxamic acid, Oxamflatin, A-161906, Scriptaid, PXD-101, LAQ-824, CHAP, MW2796, and MW2996.

4. The method of claim 2, wherein the histone deacetylase inhibitor is selected from the group consisting of trapoxin A, FR901228, FK 228, depsipeptide, FR225497, apicidin, CHAP, HC-toxin, WF27082, and chlamydocin.

5. The method of claim 2, wherein the histone deacetylase inhibitor is selected from the group consisting of sodium butyrate, isovalerate, valerate, 4-phenylbutyrate, phenylbutyrate, propionate, butyramide, isobutyramide, phenylacetate, 3-bromopropionate, tributyrin, valproic acid and valproate.

6. The method of claim 2, wherein the histone deacetylase inhibitor is selected from the group consisting of C₁₋₉₉₄, MS-27-275, a 3′-amino derivative of MS-27-275, a trifluoromethyl ketone, an a-keto amide, N-methyl-a-ketoamide, and depudecin.

7. The method of claim 1, wherein the cellular hyperproliferation is selected from the group consisting of tumors, neoplasms, and cancers.

8. The method of claim 7, wherein the cellular hyperproliferation is selected from the group consisting of gynecological neoplasms, central nervous system neoplasms, neoplasms of the head and neck, multiple endocrine neoplasia syndromes, tumors of the gastrointestinal tract, tumors of the lung, liver tumors, tumors of the bones and joints, AIDS-associated hematologic disorders and malignancies, thyroid cancers, prostate cancers, breast cancers, genitourinary cancers, neuroblastomas, glioblastomas, acute leukemias, chronic leukemias, and lymphomas.

9. The method of claim 7, wherein the cellular hyperproliferation is selected from the group consisting of myelodysplastic syndrome, acute promyelocytic leukemia, acute myelogenous leukemia, renal cell carcinoma, T-cell lymphoma, B-cell lymphoma, squamous cell carcinoma of the head and neck, papillary thyroid cancer, mesothelioma, Hodgkin's disease, non-small cell lung cancer, and colorectal cancer.

10. The method of claim 1, wherein the histone deacetylase inhibitor is administered with one or more treatments selected from the group consisting of radiation therapy, anthracyclines, flavopiridol, imatinib mesylate, retinoic acid, all-trans retinoic acid, demethylation agents, and capecitabine.

11. The method of claim 1, wherein transcript levels are measured by a technique selected from the group consisting of RT-PCR, Northern blot analysis, dot blot analysis, slot blot analysis, microarray analysis, and RNase protection analysis.

12. The method of claim 1, wherein the biological sample is selected from the group consisting of whole blood, serum, plasma, peripheral blood mononuclear cells, lymphocytes, and monocytes.

13. A method for monitoring dosage of a histone deacetylase inhibitor for treatment of cellular hyperproliferation, comprising:
(a) measuring levels of thioredoxin polypeptide in a biological sample from a patient before treatment;
(b) measuring levels of thioredoxin polypeptide in a biological sample from the patient during treatment;
(c) comparing the levels of the thioredoxin polypeptide in (a) and (b), such that continuing elevation of levels of the polypeptide during treatment indicates insufficient dosage of the histone deacetylase inhibitor.

14. The method of claim 13, wherein the histone deacetylase inhibitor is selected from the group consisting of hydroxamates, cyclic peptides, aliphatic acids, benzamides, and electrophilic ketones.

15. The method of claim 14, wherein the histone deacetylase inhibitor is selected from the group consisting of suberoylanilide hydroxamic acid, pyroxamide, CBHA, trichostatin A, trichostatin C, salicylihydroxamic acid, azelaic bishydroxamic acid, azelaic-1-hydroxamate-9-anilid-e, 6-(3-chlorophenylureido)carpoic hydroxamic acid, Oxamflatin, A-161906, Scriptaid, PXD-101, LAQ-824, CHAP, MW2796, and MW2996.

16. The method of claim 14, wherein the histone deacetylase inhibitor is selected from the group consisting of trapoxin A, FR901228, FK 228, depsipeptide, FR225497, apicidin, CHAP, HC-toxin, WF27082, and chlamydocin.

17. The method of claim 14, wherein the histone deacetylase inhibitor is selected from the group consisting of sodium butyrate, isovalerate, valerate, 4-phenylbutyrate, phenylbutyrate, propionate, butyramide, isobutyramide, phenylacetate, 3-bromopropionate, tributyrin, valproic acid and valproate.

18. The method of claim 14, wherein the histone deacetylase inhibitor is selected from the group consisting of C₁₋₉₉₄, MS-27-275, a 3′-amino derivative of MS-27-275, a trifluoromethyl ketone, an a-keto amide, N-methyl-a-ketoamide, and depudecin.

19. The method of claim 13, wherein the cellular hyperproliferation is selected from the group consisting of tumors, neoplasms, and cancers.

20. The method of claim 19, wherein the cellular hyperproliferation is selected from the group consisting of gynecological neoplasms, central nervous system neoplasms, neoplasms of the head and neck, multiple endocrine neoplasia syndromes, tumors of the gastrointestinal tract, tumors of the lung, liver tumors, tumors of the bones and joints, AIDS-associated hematologic disorders and malignancies, thyroid cancers, prostate cancers, breast cancers, genitourinary cancers, neuroblastomas, glioblastomas, acute leukemias, chronic leukemias, and lymphomas.

21. The method of claim 19, wherein the cellular hyperproliferation is selected from the group consisting of myelodysplastic syndrome, acute promyelocytic leukemia, acute myelogenous leukemia, renal cell carcinoma, T-cell lymphoma, B-cell lymphoma, squamous cell carcinoma of the head and neck, papillary thyroid cancer, mesothelioma, Hodgkin's disease, non-small cell lung cancer, and colorectal cancer.

22. The method of claim 13, wherein the histone deacetylase inhibitor is administered with one or more treatments selected from the group consisting of radiation therapy, anthracyclines, flavopiridol, imatinib mesylate, retinoic acid, all-trans retinoic acid, demethylation agents, and capecitabine.

23. The method of claim 13, wherein transcript levels are measured by a technique selected from the group consisting of solid phase immunoassays, enzyme-linked immunosorbent assays, radioimmunoassays, nephelometry, electrophoresis, immunofluorescence, immunohistochemistry, Western blot analysis, dot blot analysis, slot blot analysis, and microarray analysis.

24. The method of claim 13, wherein the biological sample is selected from the group consisting of whole blood, serum, plasma, peripheral blood mononuclear cells, lymphocytes, and monocytes.

25. The method of claim 24, wherein the levels of hemoglobin are also measured in (a) and (b), and the levels of TRX polypeptide in (a) and (b) are normalized against the corresponding hemoglobin levels.

26. A method for monitoring dosage of a therapeutic agent for treatment of a thioredoxin-related disorder, comprising:
(a) measuring levels of thioredoxin transcript or polypeptide in a biological sample from a patient before treatment;
(b) measuring levels of thioredoxin transcript or a polypeptide in a biological sample from the patient during treatment;
(c) comparing the levels of the thioredoxin transcript or polypeptide in (a) and (b), such that a continuing elevation in levels of the transcript or polypeptide during treatment indicates insufficient dosage of the therapeutic agent.

27. A method for diagnosing a thioredoxin-related disorder, comprising:
(a) measuring levels of thioredoxin transcript or polypeptide in a biological sample from a patient;
(b) measuring levels of thioredoxin transcript or polypeptide in a reference sample from one or more healthy individuals;
(c) comparing the levels of the thioredoxin transcript or polypeptide in (a) and (b), such that an increase in levels of the transcript or polypeptide in the biological sample indicates diagnosis of a TRX-related disorder.


SEQUENCE LISTING (10 sequences)

SEQ ID NO: 1 - DNA, 501 bp, Homo sapiens
gaattcgctttggatccatttccatcggtccttacagccgctcgtcagactccagcagccaagatggtgaagcagatcgagagcaagactgcttttcaggaagccttggacgctgcaggtgataaacttgtagtagttgacttctcagccacgtggtgtgggccttgcaaaatgatcaaccctttctttcattccctctctgaaaagtattccaacgtgatattccttgaagtagatgtggatgactgtcaggatgttgcttcagagtgtgaagtcaaatgcacgccaacattccagttttttaagaagggacaaaaggtgggtgaattttctggagccaataaggaaaagcttgaagccaccattaatgaattagtctaatcatgttttctgaaaacataaccagccattggctatttaaacttgtatttttttatttacaaaatataaatatgaagacataaccagttgccatctgcgtgacaataaacattatgctaa

SEQ ID NO: 2 - DNA, 529 bp, Homo sapiens
gctttggatccatttccatcggtccttacagccgctcgtcagactccagcagccaagatggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaaaacataaccagccattggctatttaaaacttgtaatttttttaatttacaaaaatataaaatatgaagacataaacccagttgccatctgcgtgacaataaaacattaatgctaacactttttaaaaaaaaaaaaaaaaa

SEQ ID NO: 3 - DNA, 421 bp, Homo sapiens
atggtgaagcagatcgagagcaagactgcttttcaggaagccttggacgctgcaggtgataaacttgtagtagttgacttctcagccacgtggtgtgggccttgcaaaatgatcaaccctttctttcattccctctctgaaaagtattccaacgtgatattccttgaagtagatgtggatgactgtcaggatgttgcttcagagtgtgaagtcaaatgcacgccaacattccagttttttaagaagggacaaaaggtgggtgaattttctggagccaataaggaaaagcttgaagccaccattaatgaattagtctaatcatgttttctgaaaacataaccagccattggctatttaaacttgtatttttttatttacaaaatataaatatgaagacataaccagttgccatctgcgtgac

SEQ ID NO: 4 - DNA, 439 bp, Homo sapiens
agcagccaagatggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaaaatataaccagccattggctatttaaaacttgtaatttttttaatttacaaaaatataaaatatgaagacataaacccagttgccatctgcgtgaca

SEQ ID NO: 5 - DNA, 371 bp, Homo sapiens
agctgccaagatggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaagacataaccagccattggctgtttaaact

SEQ ID NO: 6 - DNA, 318 bp, Homo sapiens
atggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtcttg

SEQ ID NO: 7 - protein, 15 aa, Artificial
  synthetic peptide
CYMDVIPEDHRLESP

SEQ ID NO: 8 - protein, 4 aa, Artificial
  Synthetic peptide
CGPC

SEQ ID NO: 9 - protein, 105 aa, Homo sapiens
MVKQIESKTAFQEALDAAGDKLVVVDFSATWCGPCKMINPFFHSLSEKYSNVIFLEVDVDDCQDVASECEVKCTPTFQFFKKGQKVGEFSGANKEKLEATINELV

SEQ ID NO: 10 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV